SEQUENCE LISTING (22 sequences)

SEQ ID NO: 1 - DNA, 14 bp, Artificial
  Oligo nucleotide sequence or oligonucleotide target sequence
gacgggtacatctt

SEQ ID NO: 2 - DNA, 15 bp, Artificial
  Oligo nucleotide sequence or oligonucleotide target sequence
ggacgggtacatctt

SEQ ID NO: 3 - DNA, 16 bp, Artificial
  Oligo nucleotide sequence or oligonucleotide target sequence
cagtcattgcattcag

SEQ ID NO: 4 - DNA, 17 bp, Artificial
  Oligo nucleotide sequence or oligonucleotide target sequence
acagtcattgcattcag

SEQ ID NO: 5 - DNA, 19 bp, Artificial
  Oligo nucleotide sequence or oligonucleotide target sequence
cactgtcttggttgttgat

SEQ ID NO: 6 - DNA, 17 bp, Artificial
  Oligo nucleotide sequence or oligonucleotide target sequence
ctgtcttggttgttgat

SEQ ID NO: 7 - DNA, 17 bp, Artificial
  Oligo nucleotide sequence or oligonucleotide target sequence
aacagtcattgcattca

SEQ ID NO: 8 - DNA, 20 bp, Artificial
  Oligo nucleotide sequence or oligonucleotide target sequence
agatgtttattttccttaag

SEQ ID NO: 9 - DNA, 18 bp, Artificial
  Oligo nucleotide sequence or oligonucleotide target sequence
aagacagcagatttattc

SEQ ID NO: 10 - DNA, 17 bp, Artificial
  Oligo nucleotide sequence or oligonucleotide target sequence
cagcagatttattcagc

SEQ ID NO: 11 - DNA, 16 bp, Artificial
  Oligo nucleotide sequence or oligonucleotide target sequence
tatatagtcagtcacg

SEQ ID NO: 12 - DNA, 14 bp, Artificial
  Oligo nucleotide sequence or oligonucleotide target sequence
cctagtaagccacg

SEQ ID NO: 13 - DNA, 15 bp, Artificial
  Oligo nucleotide sequence or oligonucleotide target sequence
tcctagtaagccacg

SEQ ID NO: 14 - DNA, 30 bp, Homo sapiens
ctccattgaagatgtacccgtccatgcccg

SEQ ID NO: 15 - DNA, 45 bp, Homo sapiens
ctgaatgcaatgactgttttttactcttaaggaaaataaacatct

SEQ ID NO: 16 - DNA, 15 bp, Homo sapiens
aagatgtacccgtcc

SEQ ID NO: 17 - DNA, 18 bp, Homo sapiens
ctgaatgcaatgactgtt

SEQ ID NO: 18 - DNA, 20 bp, Homo sapiens
cttaaggaaaataaacatct

SEQ ID NO: 19 - DNA, 32663 bp, Homo sapiens
tagattggtgctgagacactactgtggactcaccaggacacctaggccacttgtgctttcagcagtgttcctgagagcctgctgtgtgccaggctggtgccagaggggagggacagggctgctccagtcctgcccttggggaatgtgcagcctggttctattcccagagtcaaggacactctgcagccagttaagtgttgtagctctcttccagggcctgcaggagaggaggctctgagcacatagctgagccccagggcactcaagctgcacacttgagggaagcattgtgggagcctgtggtttttccaagaaaaaggtctcctttgggtcctgggggcagcgggagggcagaacctgaggccttggacaggtctgcatgacccacccagaaatgccatcagtttattttacaggagtccctgtcagagcttttggggcagacctcagaggagtctgtccagggcctccaagcccaggccctcaacattccaggacgcaaaatctctactctcagcactagggagtaagtaggatgcttccggaaattgagtggactccattgtctggtgccatttcctgtccaccccattcctccctagggttgcaggcttaactgtaggtgttgggtaagtcactgtctcactctgttctttactctcctgatcggtaaactggtgtagtgggtggtaaacccaatgcccacaggggccttattacattgatttcctcactctccctgttgccggctagactcaccccattcaatatgcaattggcctctcctcaaacagccagttaccccccagggaacaggtatcagtcttcctcagcctttgtgagctacacccctcactgtgcagcaatctttggggttcaagtagctctttggtagagggtgtgttcagtgactcatgataagcctgcaggtgcccacatgggtgtgagcctccaggggcaaagctggccatggaggctcagagagggctggaaagaactcgcccagctcaccacagagcccaagctggaatgagaccacgagcgtcatctaggaccagaatcacccatgtcaacagtcatctgaccccattatatacagatggagaagtgggggaggggagactcaggctctgagcagtctcttcccacagcctggcccctctgccataacctgggcaggtcccagcttggctcccacagcagtgaaggatatgcagggccaggcaggggggcaggggggcaggggggctctgggagtggggaggggtttcagtagcctaggcaatggctagagtggacaacccagttttcctgcccagtgcctgccctgccatccacggctgagctccgggcaccgagggcatggctggacaggacaagcgtcctggaagaggccctgcccgtgctggacgcctgcccgaaaaggctcgaaacccacagaagctgcctctcagtctctaagaggcttggagaggaagcggggagatgcgaattcctatctcccagttggcaacgccgaggtcgggcaggggccgggctgggtgacctggaaggaatcctccgctctgggcgcgccacgcagtcccgggtggggctgtcccgtgttagcccttccggtgcccgggacgcgcacctggcggcattcctggccaggtgtctggactgggggctgagcccagcctgtccccgccgcccctccctacctcccgggtagagcgggcgcggcgcatgtgacccagggctgggctcccgggagttacgcgctgacgccgcgtcaccccactccgggccgggcgcccattggctgcgccgggcccgcgggggcggggctggtctggctctgcgccccggctcccctgggtctccagccgctgccctggcccgcgcgcgtgcggagccgccccggctctccggctacctccagtccagacaaaaccaggggcagcagtgctgtgaggtcctgagcaagtcgcttcaccgtcccgctccacgtgcctcaatttactcatctgtaaaatgggatggtaacagcctcaaccgcctgaggctgtcgagaggagtgaatgggtttaaaccgcactgaacactctgtaagcgctcagcaagtaaactgtgccgaacctgcccgccggggtcaccgtggaccaggctggatccctgacccctgataggcacaccattacagaggggcttgtgtccacgttccttgggcgtgctcaacgctccccagccaaccggggcccgagggtcttgtttggaggtctcaggatctttgaggagagaaactgccaagacaagcatgttccccctgaaaaatggatcccctcttctgttttcccctaccctcacgttgagggttgccctggtaatcccaggctgtggggcaaagatttgtttctttggtggcaaagatgtaaattcttcccacccccacgttggactgtgccccatgggggttggattttctggggtgcaggtcttctgttgaccttgtcttaccttctttccctttcccctaactcctgcagttatggagtgaatatttattgagctattctatttacatatgtggtaacccatttaaaaacggtgaggcgggttgggtgcagtagctcacgtctgtaatccgagcactttgggaagccgaagtgggtagattactggaggctaggagttcgaaaccagtctgaccaacatgacaaaaccccatctctactaaaaatacaaaaattagccgggcgtggtagtgcatgactgtaatcccatctactcgggaggctgagggaaaagaaatgcttgaacccgggaggccgacgttgcagtgagccaagattgtgccactgcactccagcctgggcaacagagcgagactctgtcttaaaaacaacaacaacaacaacaacaacaacaacaaaaacagtgatagccaggtgcagtgactcacacttgcaatcccagcactttgggggggccgaggtaggtggatcacctaaggtcaggagttcaagaccagcctggccaaaatggtgaaacctcatctctactaaaaatacaaaaattagcctggcatggtggcgtgcgcctgtaatcccagctactcaggaggctgagacagaattgcttgaacctgggaggtggaggtcgcagtgagccgagatcatgccgctgcactccagcctgggtgacagagagagactttgtctcaaaacaaaaaacaaaacaaaacaaaacaaaaaacagtgaggcagatctgttgtgatgatgactccaaaacaccctccctgcctctgcagagggactgcagaaagggttattcccattttatagatacagtagctgagactcagaagtgaggtattggatccaggtcacacagcaagcaggtgaaaacccagatcacctgcctagctctgaagaaaatggtatttaggcttcacccagcacttcctatcccaccccctccctgggaagggctgtttaaattttcagggaagtcaccagcttcctgcagcctctagagtgttggtgggggtggggcactgaggagaagccagctttgttctctgtgttctccagcagttgttcatctggagggagtgggtcctgggtggacccttgagcagggctacttggggagatgtggtttggcgacccctatgacttctggcgccgctattctgggtaattttccaccgcagccacttctgggagaggaacaaagggagctggatgtccaggctgagccccagggacttgggctctgtggcttctctcccccacacaccccttctaaaatgcatcatgaatgttactcctgcttagggcgtggccagataggctatatctggagtttgagcaaggcagtctgcaggatggcttgacttatgaaggtctggggtcgggaaggcctgaggcccaggcccctgatgagtttcctggactgccctccaccaagggtgcttctctctgcttgcatacctctggggacagggagctcactgccttatcacattcagattcctgaatgttaattctggtttatttcaacccacctcattgggaccccttccctccttcctgccccacctggctctgtccctaggccacagaaccaggttcggtttccagccctcttctcaacagggctgcctgctctgatctagtcccagcttgtgatgatccagggcagcctggctctgatctaaagcacagctacctcttccttgcggcccctatcctggctgctcctgggaataagtgccaaatctggggtcagacagccctggggccagtcttccttgggtactggcttcctccttcaggagctgcactgggcccactggtatcctatccctacagctggatctgggaggaaaccagatgacgaaattccagcctctttctttggccactcctgtcctcaagaggccaatcttctggtttctttgcagagagggggcaggctgatctcacaggtcatgctcccctccacattgtcactagcctcccagcctgcccgtgagaaagcatcattaggcccatgttacaaatgaggaaaattgaggcagagtgatgtaactggcccagcagttacatcaggcctgctcacaacacagcaggcctgggacccctataacttggatcctggtctgtcttgttctaaagagtcaaatctaggaaatgaggaaatgaagtttgggatgggcccaggcctggggcttccactcggcttccttgcttggtgctggagaaacagaggcccagagagggggctcggcttgcccgcgttcccgcagcagccggccagaggccgctgccattgtgcgcgaggctggataaaatgaatgactggagggcgctctggaggaggggccggctgaggggagatttgtggcgcagaccggggatcaggggtcccccgctctctcaaggtggggcggggccgtctatctgggagggcgggtcctccccgaaaggccccgcctccgcctcgaccgcccagcagagctgcggccgggggaacccagtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaaggtgcgtcagggcgggcagggcttgaagctgcgccgggtggcgcgagtagggggcgcgcaggtgtctccctggcctttgtctcccccacgggcgccagctccgtgctgtgctcgcgcgggacttcccggtgtctctgagctcggtgtcccgagcctcaccgagcctccctggttcccgcgctagcgtctcgggccgcgcgcttgtgggtgagggctcctgggccgggccggggtcccttggcggctccgggccgggacacgtgcgcctctacgcgtcccaggccgggtgccgcccgaccggtgactctccagccctgtgatggccacggctgaagctggggacccaggcgtcgccgaagctccgccccagccccagccgtgacgtaattgcgaggttactcacggtcattccctccggcccgagagttcagctcggcgtcggagctcttgcgcatgcgcatgggcgctgcctcgcgcccttcccccgcctcgtgtcgggttctcccggtctgcgacgggcacagcctccgcactcattcactgacatccaccgaatgccaggccccgtcttaggcaccgagggtttacagacagacctgggtaccccctcttttagggaacacaaaaatctcccgggaaaccaaacgggtatttagttgtaccttgggtggagcgaggctgggggagggcagggatgtggctactttgggtagagcggtcagggacttctaagctgagacctgagggtcacccccaggaccagcaaggaaagatgttttccaggccacggcaagggaagggcaaaggcctcgaggcagggcctaagtgtgaggagttagaggcttgcaaaggagtgaggtcagggaggaggaggacgcaaaccgacttggtcggccagggaaagggcggagcagaacagtggcaccggcttccatctttggagcatcaccctggctgtgatgagaaggggtttggggccaatggtggcaccaagtgccaattaggaggcccgttgcttccattttgtagatagagcaaacggaagcccctagcaaattgcctgcatggtttctgtgcaggagttttagcagcactagctaagttgcacttggttgatgaggaaactgaggccaaggtcgcaggaacaagatgcctagactcacagcctgaatggacatgtccatggaacccgtggccaccctggggttggcaaaacagatatatctatgccaccaccactcctgccctactgcagccttgcagatgagcccagctggttgccagccccagaagcttcccagccctccctccttccccctggggctgggctaggggaggaccccagaggagaggccctgattgtgaggcttttccaaaacagcctcccctatccctggcacgaggggttgtccttcactgccctctggagtgatgaaccctgaaatcccaagccctagggagatctgggcctgactcaactaccagttccacatcactgggcccagtgagtgtagtcccaagaggcaacgtgaccaagccaggaggacatgcgctttggggtcagaacttgaacctggacactcctcacttcctttgtcatcctgctcaagccctctcaccctctaaaccttagtttccacctccagaaaaatgatgcaaaccctcccttcatgggcaagttggacaacagaacccgttctgggccacaggtctgatacagacctttgtttgtttgtttgtttgttttctgcagtggcgcaattttggctcactgcaagctcctcctcctgggttcacgccattctcctgccttagtctcccaagtagctgggactacaggcgccagccaccacgcctggctaattttttgtatttttagtagagacggggtttcactgtgctagccaggatggtctcgatctcctgaccttgtgatccgcccgcctcagcctcccaaagtgctgggattacaggtgtgagccaccgctcccagcccagacctttcttactgacagaatctggtctgggccagaggtctgatacagacctttcttactgactcatggataaaaacattgtctctccagaaccaaaggccaggcatgggcagccatgtggcccaaggtctagtctatgagagagtgggggcagtcccagccccttgaagactgggggcagccccttctcactaggcagggctcagctttacccacttcagtagaggatttttcagtttttattcaaacttcctgtttttcttcccaattacacacatcttttttcattgtagaaaacttagaaaatgcaagtgagcaaaaagaagaaaataaaatctttagacctggggtggtggctcacacctataatcccagcacttgggaggtcgaagcaagaggatgacttgtgtccaggagtttgagaccagcctgggcaacatgacaaaatcctgtctctacaaaaataaaaaattagctgggtgtgggtgacatgtgcctgtagtctcagctactctggaggctgaagtgggaggattgcttgagcctggacttagaggctgcagtgagctataaccatgccgttgcactcagcctggatgacagagtgagattctgtttcaaaaaaaactttaaacctaccacccagagataagccctgctaattatgtgaaagagcttttcttctctctctctctctctctctgtgtgtttatatgtgtttggggatgggtgcacactcttcataaacttttttttttttgagacagggtctcgctcttttgcccatgctgtagtccagtggcatgatctcagctcactgcaaactctgcctctcaggttcaagagattctccagctcccaagtagctgggattacagtcatgcaccgccacgcctggttaaattttgtatttttagtagagatggccatgttggccaggctggtctcgaactcctgagctcaggtgatctgcccacctcagcctctcaaaagtgctgggattacagggcatgaaccaccatgcccggccttcatcaattttttaaaaacgactttattgaggtatactttatgtatcacaaaatttacccatttttagtatatcattcaatgatttttagttaactttttgagttgtgtgacaattactagctgtcgaacatttttatcacacagtgagatcccttatacttctttagtcagttcctgttcctgctcccagccccgggcagctgtggatctgtatgtgtgtgtgtatatatatatatatatatatatatttttttttttttttttttttttttttttttgagacggagtcttgctctgtcgcccaggctggagtgcagtggtgcgatcttggctcactgcaagctccgcctcccaggttcaaacagttctgcctcagcctcccgagtagctgggattacaggcacctgccaccacgcccggctaatttttttgtatttttagtagagatggggtttcaccatgttagccaggatggtctcgatctcctgaccttgtgatctgcccacctcggcctcccaacgttctggaattacaggcgtgagccaccgcgcccggctggatctgtatttttataaattaaaatagggtccattggttcacagctgattggaatctgcttggttccatgtcaacagccagacgacagtaaggtttcctcttattacccacctgattccctgtcgatggacacctaggttgttttatctttataaactgctgcagtggacactgaggccggtttttttctttgtttttttttttttgtttgtttgtttttgagacagagtcttgctctgtcacccaggctggagtgcagtggcgcgatctcggctcactgcaaactccgcctcccgggttcacaccattctcctgcctcagcctcccgagtagcttgggactataggtgcgtgccaccatgcctggctaattttttgtatttttagtagagacggggtttcaccgtgttagccaggatggtctcgatctcctgacctcatgatctgcccgcctcggcctcccaaagtgctgggattacaggcgtgagccactgtgcctggccactgaggccagtctttgcccggatcctcactgtgttcctaggatgaggttctgggaggggaattgctggtcagaggtcgagcctgcttttgaagcttcttctaccaggagtggagctgagcaggtttgataaggtctgaagatttgggggtggaaatgccaggtcccttgagagacatgagggataagagggggccaggctggccttgagtgccagagtgcagagctgggctagatgtgaggacagtcgggggtcagagcaggggcacaccgagcttcagttccctctggctgcttggatggaggatcgtaatgtgaacagaaaacactaattgagtacttactgtgtttcagacagtgtgttgataatcccacttaatcccctgacaaccccaagtaggtagacatatgatgaagatgacggccttgaggaccagagaggttaagtgatttgcctgagatcacacagccagatgatggcaaagccagaattcaaacccaggctgtgggctccagagcctagctcttaagctcttaagcactgggctcctaagaatggggatgaggggttgagggaggctcctccacaggggctactctgggggcctggaagtgggtcacagaggggtcagaggctatgtggctacctccccatcccagtccagagcagtgtttgagtcattagactgggaaccagccctggtgagccagccaagggccttgggccccatccggtcctgctgcctgccacagccaaactcttgtcatgtgaatggatttggggatggagctgcctccatgagtccttgcatctgtgggtgaaggcactgccctggctatagtgtccctgggtttgagtcctgcatctgcaccaagacctcaggtgagcctgtctccttctgggcctcagagtaccttgcagctgtcgggggaggatggatcaggagatggccctgtacctgtgttggggattattgttaagcccgtggcagtcttcacctccctgctgaggattaatttatccaattttgcacaagcttatgagtgcagaagaggcagacggaaacagagttctggccaagagcctggaacagggcctcggggtctctttcctatgcctggaccccgtcatgtctgctctttgtctgtcggaccccagatgtctgccaagccccgtcagaggctgcttcccagaaagcccttctgggtgtcaccttgccccgagcagtgcgttctcagagttctcccgccctgatgtccctcccagcatgcccagcccagccacaacagggccttgcttctagtcatgtgtctggctgtttgctgggtccaggccagccctggtagggcacaatgggggcccgctctgccaccccatacctctccccaggatatctcatgccccagttctctccctagttccaccaagcactggcactccttagaaaacacagctctagactagttactgccctagcttacagcacagaactcccctggtctccaaccattcatggctccctagtgctccaagataaagttcccttgtctcagccgggttgggagttaccttctgcccaacattcacctagctggacacaaacatcctgagtgacccggtcagctccaggcaggagtcactgccagcagaggcctgggatctggactttgcctgctgacaggtggagcccaggccggccagaggaagtgcctctgaccttgtctcctagcagccacgggccatgtggacatgccttttgaccctgggcactgacagtgtgtgacagcctgcaccatgtgctccacaggggcggctgtgtgtgtcgggggtgaggtggggaaagccttaactggctcaggggtgagaggtcagggagccattgagactggctccaggtgtgggtcccctgctgggttggggcttgtgggaggtgggacggggctgggggtccatccccctagggggaatttgtggcctaccccgaaccctgtttgagctcctttcctaactgactccccgtccctgcacctgtctcccagcaggccttgcctctgcatgctgcccctgccaggctctggggtccctgtgctccctgcagctagaaggctgggatcaggggtcttaacaagcagccctactgtatgaccttggacaagtccaagaaccttcaggttcttaacaatgtaaagggagcagtactaaaagcagcttcttggaattgtggggatccgatgagtgaaggcttaagcagtgcatggcacatagtaggccctgaaccaatgccagttagtgttattattatcaccatttagccagatgcagtggctcacgcctataatcttattgactttagaggctgaggttggaggattgcttgagaccaggagttcaagaccagcctgggcaacatagcaaggccctgtttgttttagagaaaacaaacaaatcaccatttagagcacctaaccagtacctggcacgcgataggtttagctcaacaaatgttagcagcaattacccaaggagcctgtgctggaagtttctaggatgtaccaggctatggttccaagttctgagcatctaccatgtggtggtctggagttggtgagagacaggatggggctgactaggccagtggggagcaccccccgccatggggaacaagcaccctatccttggcttccatggaagataattgatgctgggcacagtggctcacgcctgtaatcccagcactttgggaggctgaggcagggggatcgcttgagtctgggagttcaagaccagcctgggcaacattgtgagacccaaactaaaaaaattagcttggcatggtggagtgtgcctgtcgtcccagctactcaggaggctgaggctaaagctggaggattgcttgagcccaggaggttgaggctgcagtgagccatgatcataccactacactccagcctgggcaacatagtgaggccctgtctcaaaacaaacaaacaaaaagaacctgctgaggaagcagtgtttctggctgggggaggacgggcagagtggccatctggccacagatggcggtttctgtgcaaaacacatcaaggcagccttggaaatgtgagtgaaagcaccttcaaagttctggtcacagccttgggactaagcaaagccaccaaaagtacataaaagacaatgaccatcacccagtgccggtgatgctagaaggaaagggaatacgttgtagggaaggttgtaaagggctttatcttttccagactggagcctggcagctcgaaaacatcttgctgccttcatatgagctttaaaacaagctgcagagaaacaactcaagagggagaaatatatatatatatgtgtgtgtgtgtgtatgtgtgagtgtgtgtgtgtgtgtgtatacatatatatatatatatatatatatatatatttttttttttttttaagatggagtctcgttctgtcaccaggctggagtgcagtggtacaatctcggctcactgcaacctccgcctcctgggttcaaatgattctcctgcgtcagcctcccaagcagctgggactataggcacataccaccacgcccagctaatttttgtatttttagtagaggctggatttcaccatgttggccaggatggttttgatctcctgacctcgtgatctgcctgccttagcctcccaaagtgttgggattacaggcgtgagccagtttgtttttagagacggggtcttgctctgtcacccaggctggaataccatggcacaatcacagctcgctgcaatgttgaactcccgggttcaagggatcctcccacctcagcctccagagtaatggagactacaggctcatgccaccatgcccagctatttttaaaactttgtagagatggggccttgctacattgcccaggctggtcttgaactcctgggctcaagtgatctgcctgcctttgcctcccaaagtgctgttattacaggtgtgagcccctgcgcctaaccttagcactgccattttgactgaaaacaggtgcccagcagcaggggctactcccagaattgccactgcatcaggcccgtgggttgttttcagctgccagtgataagtatgtgccctgggccacctctcggacaaggtgtctgaattggtgccgaccagcatcacatgtaattgccatctcgcaggtgctgctgagggtaattccgcacacctgtagctccgggaagagcctagtggggaggaggaaacgtggctctgaggtttatagggtcagacggtcagtatgttgggagctggcatgtggaggggcacagacaagggaagaatgggaggtggcatcagagcaagttttgatggaggaataggaattcaccaggtggaaagggcattcctggtggagggaacagcctggccttcaatagcttgtggtgttcagaaagcaggcagggaaagggaggcccagggagacaccagttaggggatgggggtggaggcagacgagggtggaggaagccatggctggagtctgcacggcctctgactggggtccctgctgtggtcagccctgtgctgggtgaggctggggtcacagctggttcaggccctgacaggaggggcccccagctgaggcccagcctctaatttggcagggcaggtggataggtctgggggggtggtggttaggaagcctccaggaggaggcagtgccggagctgagccttaaagagcttcgtgttgtcctctctgtctttgcactctgcacacactcactgaactgcgacaaatgaggatagctggtcagggcagaggcaggccggagttggggctcactgctgtcccccacaggctggggctgaagggcaggctctggggccgcagaatggggtttgtgtaccagattcttcatatggcagctgtgggactttgggcacgaggcctccgtctgagccttagtttcctcaagaggacctgcgcccaggtgcacctggggctccagccatgggtgcgtcccattccgggaagagctggcacacacttgtgcccccggggcagccatgagtgcacaaagggcagcctgtgccactgctggatacacgaccagctgagaacacgaggaccgccgactccagttaggaggatcaaggaagtgcctggtgggagcagaacagcaggtggggtgcagcccagctccctggagggatggtgggcacccatcctcaccctgctgcctccattagcaggccgagagggtgtgctctggaatcccatgagcacctgtgccacatcctcccctgtggctgacccttcttcacagttggtgcagctttgtggtctgtagtgcagggatcaattggcaaatccctttcccacccattccctggagaattggggtccttggctcagatgacagaccaacctgagttggaatcccagctccttggtggccgtcctggcctccaccccctcactgcctccgctcctcctatcctgcccacgcccactgcagggcctttgcacacactgtttcttctgccctcccttccggcccactccctcatatcattcagtcctcctttcagatgtcacctcctaagatgggctgccctgaccacctcatctataatggccccagtgcctggcacaggattggcacacagtagatattgtcagagatggatctgggttctgtggacaaggctgtgggggcaggtgaagagctccctcttccaggaggttgtttggggttcaaggccttgtttgggttgtaggcttctgtgctggtcagcgttgggccctacaagcgcatgccatgaggcctgcccaggatttccctcatggcctcacagaatacatcggccagagtcattaaagggcgcctgcatctgccttcagagagaggtttgaaggtagaactggggagggatgccaggtgggggttcaggtttcctgttgggtcctgatagaatcagggcaggagaggaagaagaagagggaagaggaggaacccaggcttggggaggggtggcagggcttcacaagcctggggaaggtgaactagggagcagttggggccaccatggcccagagtctatgcctcctcttccttcctgtgttcagagtgtgtgtgggaaccacaagggccttctcagtgttcatagggaagcccggttcacccatgggtgggccgcaatttgggtgccacagtgagcccctagagaccagctctcccagcttccaggacagggactaggggaggcaagagaggctcttccttaaattgtgcacccaaggtgcctcagctgccttactctagactggccccgttaactccccttaaaaaaaaaaaaaaaaagactcagtcgaatggtaatggagctccaacgtgaatactgcaagtatcaggcaactcactacctgactttccagttctaaaccattctaattgctgtagagagaactaacctttgttgagactgttgagtgatggatgttttacacacttgctttcccagaattcccacctctggagatcgtaggtgtgggagctcagagggtggggagtggactgtccccatcacacagcaagggaggggctaaaggaagagcagggcctggcatgcagccccagatagcccacttgggtgtgtctctgagggaggctgcagggctggctctagagtttcctttttcagtcttaacctggtgaccagcttccacagaaattggcacggtgactcatgcctgtaatcgcaacacattgggaggccgaggtgggaggatcacctgaggtcaggagttcgagaccagcctggccaacgtggtgaaaccctgtctctactaaaaatacaaaaattacatttcattacaggtgtggtggcgcacacctgtaatcccagctactcaggaggctgaggcaggagagtcacttgaacccgggaggtagaggttgcagtgagctgagatcgtgtcactgcactccagcctgggtgacagagccagactctgtctcaaaaaaaaaaaaaaaaaaaaaagaaattggccagtagatcagccccaggggagagtgagccagggtttggccaggccttgagtttcagaggctggccatggccagtggcacccaggcccttcccccttcctcggggcatcttagcttagtctgtgccctctgcccaagggccagccctctgttcccaggtcacaccccctcctcttggaaggccccccccgccccacccccatcagagtctttaatgactctgctgcccctggggctcagagagcaaccgccctctcccatcgcgcttcctcagtgggatgggagggggttagagcaggaagatgagacaaataaagacacaataagaggcaggaatatgtggtaaagccaagatgggtaaggggaggggacaggcttgactgttcacagtggccctggccctgctgtctcaggctagtatctgcttgttggtctcaccacattctaggctcagaaactggggagcaaagtaatgaaagaaccaggctgggaggccatggggaactcatgcctggagttcagctctcagtgtgcttttgggtcaaggacgcttccctgtcttaagtcactcatgtcagagcctttgccaagagcaatgctgtgttttgttttgggggtgagggaacacccgcgggctgaggggagggttgggccatgctagagaggccgtctgttgtccttgaacctcccaaagctgggaaataagggcctgggctggacggcggtggcgaggacaggttgcgagagagacatggctgggttttcttgcttagggtcctgaatagagagcaaggttgaggccgcagggaccccagcccccaatggactgctgagtcgctgggtctgcccagggttcaggcaccctctcaggttgcagccaactggggtgtggaccaggcagaggcgctggcctgcagtttggggcagaggcaggctttgctggtggtctacttggctgcaaaatcaactggccaggctctgatcactttgtgtgtgtgtgtgtgtgtaacttttacctttgacaaaagagggaagacaggcccaggcacctcctcaaaagaaccctagagcctgtcaccccttccttacccatcttctgtcctagggactgcagcccttcctggcttcccagggccctacaatgaatagtgggtcgggactcacttggtgactgctgggttgtgaggccttgagggggaggggcagacttcacccatctggcagagggacatcggtgctggcagtcaggaaacccttatttccaggcctcagtttcccggaagtgacctgttttcaggagtggcctcatcccagaccatcagccccgctgtggtgaggggtggccccttcctggggctgccctagaagggggaggtccctgcacccaccgcagctgccactcggcagcccttggccttaattaaacgcttcttgcgtactaagtgctgcacccatattatctcccttctaccattcgacgccagggagataatgactgtcctgttttctggaggagtaaacggagggttggagcggttaaggctcgctcagggtgccagcgaaccagtgatttcgaacacagagttctggtgtgttgggccaggacttctctgctttgaccctttaacgaagggggcgggagctgagggccagtgaccgccagtaaccccggcagacgctggcaccgagcgggttaaaggcggacgtccgctagtaaccccaaccccattcagcgccgcggggtgaaactcgagcccccgccgccgtggggaggtggggcgggggccggggccgggccctagcgaggcggcagcgcggccgctgattggccgcgcgcgctcaccccatgcccggcccgcagccccgaagggcggggcggggcgggacctgcaggcggggcggggctggggcggggctgggggcggggcggggcggggcgggcgcgccgcagcgctcaacggcttcaaaaatccgccgcgccttgacaggtgaagtcggcgcggggaggggtagggccaacggcctggacgccccaagggcgggcgcagatcgcggagccatggattgcactttcgaaggtatttttggaggcctccccaccagccctttatacaatgcctccgtctcctgcaggttctcctggggtgggcgggcatgcgggctacgcaacttgagcaggaaagagccccttcccgagggagaaggtgtgacagttaccagctcgctggggaagtggagggctacctccaaccaaattagtgtcccctgcaactcaagggggaagggtttgcttagagacccaaaagcagcatcccgacctaagagggtttggagggagagggtggtcttctctacattctctgcacccgctttgggacaggaccaggaggaagcagggaggagggcccgttgtccctctgccacagcgtctgccctattcagcacccctgcctattgtgggcatcttagacttttcaggaagacagtgggagccctagattgtcaaaattgtcagtttttctttcaggcctcagtttcccccatctatcgaagaggctcacacggactggggtaaagggatgggaaaccctgcagttgaaagtccattatgacttgatgacttgtgacctggggggtccacaaaccaggagagtttctacttgagaagccaggaagactggggctgccaccccatcctgttctgccaactgctctaggaaattcccctcctgcagtagcttccctgcctgggtacctgtcagtaggcaatgttgggtctccactcggtgccagctgcctgccaagcaaagcctcgggcagccgtaccaaaaggggtttagtcttttctgttgtacagatgaggaaactggggccagtgagaggaggctgttggtccaggctccacttcaagctggtggtgggcagggctgggagctcaggctggggatcctgagagcactggaggcccccatgggtcctgtagagcattctgacccagtgggtgccaccacgagtgggttagagggccctgggctgagccagataggctgctagtcaccagctgggggagagggcccttggccaggtggggctgaggtgggagtgtgtcccagtctgtatgaggaggaaggagtcaggacagacagcacttgcttttacagagatgaaatcaaagccctgagtggccaggcctgggtcttgaggctacttggctgcaggcaaagcctggacttgagcccagaactctacacagagacacactggttggccatgtggccagcagctggcttggccctaagccttggtctgttccactgagtaatgggttggtgatggcagcctggctcttggcttcttagtggggcaagaaaaggcagagagacaatagatttgggattttgtagacctgggtttgaaccccactgcatgctcttgggctgcttgtggtcctccctgagcctcagtgtcttttcttgtctccaagatgaggtgagctaatcttttgaggtagtctagggtagtggccagtggttggggcattggagtcaaaatagggtctggactcagttgagtctctgactctataagaacttaggccagtaagtcacctctctacagctcagtttcttcacgtgtagaatggggccaatgatcacatcaccctctcagctgtgggtgaggattaggggtctagcctggccccatcaatgtgggtagccccacagcgggcctggcttttggaccagacccacccttctgacatgggcccccacccttagagtccttctagtgtggatgaggaccctgctctgatctggggtcctcttgggggacttccctgtctgccattctctttggggatcctgcgctgccctaggaagagtgggcccaggctgcacagttggtccttggtcacagaggatcccaccacttcttcagggcctcaaggcaatcctgcctctctctgcacccctcttccccctgtaaactgaggggaggggaaaatcacccactcctcagcagtttctaagttgctttgtcaaattcagtgcccagaggatcctgctgggggtgcgttttaggatgagaccaggagtggccaatggtggggtgtggggcccatcgctcctatatgaagaccccctctgccctagactgctcctccctccccatccccatctccatcccaaagactggagctgctggatctgtggatggaggcgtgccccccgtttcacacattgagaaacaggccccaagtggagccagggaaggctgcacctgggcctctggattccttttgttctgtgtggggttgggggtgatggactgtggagagggcaggagagctgtctggaagggttggtcacctcatgggcaaatgcttggaagctggtctgagtccacggtgcagtgtgtatgtgtgtgtgtgtgtgtgtgtgtatgtgtgtggactcagaggtggatgtcttgtagaatgcatgccccatgaagacaggagtaaaagtttaccaccatccacatcaagctacaggacactcccagctccccagaaagttgcttagttctaggcagggatttcccttattcacagccgggagcagtgcctggcatagtgtgggcactcagcactcagcacatgctcactggatgagtgaatgaatgtgagcctgctgtttgctgtggactaaggatgtttctagatgtttgggcaaataccggatggtgggaagagctcaggctctgaagtctgcagtcttgggcccgaccctgggctcagccccagcctagctgtggggcaagattgtgagccttgtggtgcccaccttgtccaggtattgtgatgcactcgcagcagcaggcattgctttagacagcacaggtgctcgcaaaatggctgtatgtccgggaacaccagctcctgtgggtggctttctgtcctggtggcattgcccacacatacagctgtgtgccaacaagggttgtgcaaataaggttgtgtttggatgtgtgtgatgccctgtttgggggtcagtctctgcctcactcacgcaccctcttctccttttcacagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggtaagggggatgtgtggcgggaggggacacccggggtggggcttccaggagcacaggaagaagcttctgctgtgatgtgagtagaggtctgtgcaggctttagaaactggggctccactcggctgcttgagatgccctgttactagcagtcctggtgtgcttgttgccggggtaggcgcaacctcgcactggaggcctggcttgaagccagtgcatttgcatcagagcccaggcagggactgtccataggaagccacatggggcaatgactcatccaaggccagtcggtgatagagacctgaagagcaggttgaaagtgggagagggaggtctgtgtctgcagccccatgctttatttctgcaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtgagggggtctggccaggggttggggagggggcagccccagcccagacacacagcttacagccaagcctctcccaccctcaggtcctgctgcagccccacttcatcaaggcagactcgctgcttctgacagccatgaagacagacggagccactgtgaaggcggcaggtctcagtcccctggtctctggcaccactgtgcagacagggcctttgccggtgggtgacgtgggcagggcataagggagtggggtctacacacacacacacatgcccacctggtaacatgtgcctggccctgcagaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaaggtgtggagaggcctgcaggggcacagaccggggtgtccctaggaaggaacagatcaggggcaactggaaggaagagagggagtgagactgagcctggacaagcagggaattggaattcagcctccccaggcctggccagcctcgtttatttagttaaactggtttgcaggcctcttcaataaaggtggggctgtgctaggcattggggatgcagcaatgaacaagacagacaaaaattgtccctcaaagaagagccgaccttctggtgggggagatggacagtaggcaggatgaataagtgctcgagaccaccacgtttggctcgttgcagagaaagcaggaagaggatggtgagggtcccctggtggtagccagggaaggcctccctgagatggcggcaggcacagcagcagctagccagaccctgctgtctgcatcttacattctaaccctatgcccggcctgggaggtgggtgctactaggcgaggaacggttcaggtagaaggaacaagtgcaaaggtcctgaggcagtaatgttgcaaagcagctccgcacccccttgctagggctctccaaccccacaacccccgacctgacaggccacctgtgcgctccccctccctcccacaccgtgcagctgaataaatctgctgtcttgcgcaaggccatcgactacattcgctttctgcaacacagcaaccagaaactcaagcaggagaacctaagtctgcgcactgctgtccacaaaagcagtgagtcctggctttattgagctccagtctggcctcttctctagccttgctccacctcccggccccaccccatccctagccccaccccacccttggttctggcccaccctctgccctgcccacctcacccttggctgtagccctgcattcagctctagtcccttggttacctctggtcctgaaagagacctggtgcctccctttggccctaacccagccccatcaaagcgtcctgggctagctttaggagctacagtagtccctaggcctccaagggcctaggctctgatttggggtcacatatccagcctttactcctggctctgttcctttcggcccacagaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggttgggccctgccacggtgcccccttccccactcccagccatatcctctgagcctcatgacagggccgggaagaccctaacagatcctacctcccatttcatagacagaataactgaggcctggagccacgtggggtcccacagtaaggtgggcagaatcctgaccccccccttcccagccccatgctctctggggtccctccgattctgccctcaccaccctgcccaaccccaccaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagaggtgggaccggccagcctgggcatctttgggagggacactcggggtgagcccccaggcttgtgaacttggggctctggatttcctgggagctgtgtccccagctttccctctgtccatagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccgggtaaggggctggccccggcagagtgggcagggcagggaccccaggctgtgaaggtgctgggtgtcaacccttgttcctgctccctgtgcacaccatgaatctgtcccgtcctccctgtgcctagccacgcatccgcagacccccaccacccctccagagcctgctgtggacggctcttctgagctttggggcagctgctctgacctcacttttctcacctggaaaaccctcatccacagggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatgggtaggactgagcgtggggcgggctccgaggtgctccctgctgcctgtgctccacccacagcctcatgcctgcttgccttccagggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacagtgagtgggttggggggctgggggcttatccctgcagctctctccagaggctccctgggtaagagctacacgggatgtggcagtggttaccagggggactccaggccaagctgggactcggcccggggtctggccccaggctgtgtccactgtgacagcccagtaccctcccctacagcgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccaggtgctctcttaccccttccctgtcccctctcctgtccctcatcctcattcctgtcctgtcccttgtcgcctgaatctctggctgtctctggccaccccagtccttctccctgccatgggttgttgctgtgggggttgcaggaagggaaaggcctgggtgcctctcgttcccattggggctttcagaagcacatgcagggattgatgggcagatggctaattggagaagtgaccccaggcagtgccgctgtggagtaaggaagcggagccaacaatggcatcttctcaagtcggttttcctttggaagcagtgtagggcaggcctcagtgttgtctcctggccaaggctggtgctggtgatagttatgtccacccgctttcccctgtccttggcaggggctgcacccaggggcatgccggcacttcccagtggccctaggtgtggccccagcccacccaggaaaaagcccttagcttggagaggagggtggggccctgctccccaccccactcacctcctcctctccacagtggaccccctggcccaggtgactcagctattccgggaacatctcttagagcgagcactgaactgtgtgacccagcccaaccccagccctgggtcagctgatggggacaagtaagtgtcgttgtgccctcctccaggcaaggcccctccggcgggattctgagaatagctctggcctcaaccctgtggagagagcccagagctgggctaccgtgcgtgccatgcacgcttcattcctctctgagtttcctctccccaccagcctgtgggaggagacagtggcactttgcagagccaggggccaggctgtactctggagggcaggtggggagcaccctcctaggacccctgccatctgttccgacagccagctctctccttccacagggaattctcggatgccctcgggtacctgcagctgctgaacagctgttctgatgctgcgggggctcctgcctacagcttctccatcagttccagcatggccaccaccaccggtgagtccccggcccctgtcctggctcccttctcagctcccccgtgcagcgtgactgagggttcaggggaccctccctcttctgcaggcgtagacccggtggccaagtggtgggcctctctgacagctgtggtgatccactggctgcggcgggatgaggaggcggctgagcggctgtgcccgctggtggagcacctgccccgggtgctgcaggagtctgagtgagtgcacggcaggttcctcctgcctggtcccgggctcagccttcctcatcccctgggcactgtgcctcactcagcctttgttctgtgcaggaggagtcaccaccttttttcctcagggaactcgagccagggaagtggggggcactcagccagggcttgtggactggtctgactggcactcttctgccctggtcccaacaggagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggtgaggggtggggtcaggggcctggcagggctgggggattcagctttccattccctggttcctctccccagcccccaggggctgcagaagaccatggggttagcccaagcagcacaggatagggggtccagcagaccctgctttttggctaaggcttctgtccagaggagaggggttgcccctatctggcctcagtttccccatccctgggaggaggggggtggatggtgtggtaggatccctttggaggccctgcatcaggagggctggacagctgctcccgggccggtggcgggtgtgggggccgagagaggcgggcggccccgcggtgcattgctgttgcattgcacgtgtgtgaggcgggtgcagtgcctcggcagtgcagcccggagccggcccctggcaccacgggcccccatcctgcccctcccagagctggagccctggtgacccctgccctgcctgccacccccaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgagtgcccgatggccctgtcctcaagacggggagtcaggcagtggtggagatggagagccctgagcctccactctcctggcccccaggtgttcctacatgaggccacggcccggctgatggcgggggccagccccacacggacacaccagctcctcgaccgcagtctgaggcggcgggcaggccccggtggcaaaggaggtgagggggcagctgctgaccagggatgtgctgtctgctcagcagggaagggcgcacatgggatgtgataccaagggaggctgtgtgtgtgtcagacgggacagacaggcctggcgcagtggctcacacctagcactttgggaggctcagttgggaggacagcttgagcccaggagttggaggccgcagtgagcctgagtgacagggagagtccctgtctcaaaaaaaaaaaaagaccaagcatcttcttgatggttacctgatgacaattcctttcacaaggaatcagtggggtgactgtcatttgtgggatacatgactgcacgtgcgtgactcagtctgtggactttgtgtgtgggctgagactagggtggggagaggggaacccgccaggcccccgccaggtacctgtgtgccaggtacaggcggctggtgccgtggcttgtgtgtgggcagggctcccgcgggggcgtggccagcttgagacccatccctgacacatcctcgtgtgcgcaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctgggttttgtgtcttcagctgatggatgtgctgactagtgagagtgcttgggccctcccccagcacctagggaaaggcttcccctccccctccggccacaaggtacacaacttttaacttagctcttcccgatgtttgtttgttagtgggaggagtggggagggctggctgtatggcctccagcctacctgttccccctgctcccagggcacatggttgggctgtgtcaacccttagggcctccatggggtcagttgtcccttctcacctcccagctctgtccccatcaggtccctgggtggcacgggaggatggactgacttccaggacctgttgtgtgacaggagctacagcttgggtctccctgcaagaagtctggcacgtctcacctcccccatcccggcccctggtcatctcacagcaaagaagcctcctccctcccgacctgccgccacactggagagggggcacaggggcgggggaggtttcctgttctgtgaaaggccgactccctgactccattcatgcccccccccccagcccctcccttcattcccattccccaacctaaagcctggcccggctcccagctgaatctggtcggaatccacgggctgcagattttccaaaacaatcgttgtatctttattgacttttttttttttttttttctgaatgcaatgactgttttttactcttaaggaaaataaacatcttttagaaacagctcgatacacacaatcttcagtgtgaagcaatatactaataagaacactagtcgtcttaacatttacagtcttcatatatattatatatatgtatatgtatacatatatatacactatataacgaggccagatataatacacacgtttaccattttacagtcatatgtacaggaagttgctagggcggccctgggctgggggctgcgtcaggcctatcgaagcgtggacagagctgaggacacggacggacaggcggacggactggcagggactggcccgggccggtggtggctgcgtggacaagtggcgtcgcggtagccccttacccggcaaaggcccggttggggctctgttgcgggcgcacgacgcacggcggcgacacacgggagaagcgagcatgttcccaacatatatacattctatgtaacatatatatagaggggtacacaggttttgtacacgaaatctagcgctgtccctgctcccgtgcagagtaggcgcggcggtccctggtggtgcacaacggtcgcgcgtccgccggagccggaggtgcgttctggccgcttcttgggttttgcgctcccaggctgggctccaggggtggggtggaggactggaaaggggacaaacagaggccaaagggtgtcccagtcccgcccacactcggggatcccgcaacccctaactgagcatagcccaggtctaccccggctttgcccgaaccgcacgagagatctggcaaaggctgggcgggaaggcccctggacgcgttccgcacggtttgttccaaaggctccggcctctccagtgccggcctagcccgctctcagaaaggcttcctttctcccccgcctagtccacggagctaggagccgccctccgcctcttccagtactgtttcgacccccacccccaagcccagggctcccagctgcacgccacgaccgactggggtggcggcagcacccacgtggaatgtgtgccacgtcgtcttgagattccagagggaaagtgtccccaggacaaagccgtctcccaccctcgccggaggctgagcgccccggggagtctggccgctccgcgggctccccctccgcaagcctggcccctggggtgggcaagaacgccctgtgccgtccggttccgggagcaggcggctccggaacgggttttcctcccaacccggggccgcaagtctgagccctaggcggccggatccacgatccgggacccggatcaaggatcggctctggaccagcgctgggagcagctcaaaggcccgggggctgcgcaggcggctcctctccttcggcggcgggcgggcaggcgggctccggcggctcctccggccgttggggtggattactacggcagcctctgcaaaagacgcccgcccgccagttaccgacgacctcggacttcacagtccataggccccgcccccagtgtgggccccccgcctaagccccagacccaggcgaccccagagcgcggctggggtctccgcccgaggccgtcggcaccaccccttctggccccaccccgtccagcttcaccttcaccttcgccttcagctccgtgccaccgtcccgcgcccccgcctgccccactctgtcacctatcttggggagctgcagggaggccgtcccagggacgccctccagcccctggctgcccatgccccgacgcctgcgggttttcggcggcggcggcgcgcaagtggacagggacctgagctggacggcggcgggggctcctgggggtgggcggggagagctccctcccctagggctgggccctgcggggctgcgcagccaagggggcccaggggactgtgaaggaggtgcgaggtggggctcctctcccgcttgacacccctcgccctacaaaacactgtggtcccctaccttatgtttgggacatttcaaagaaaagttctcttcgatgaatatgcaacctgtgaccaaagagaagtttcggtggttaggacagggtggg

SEQ ID NO: 20 - DNA, 5012 bp, Homo sapiens
agcagagctgcggccgggggaacccagtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaaggtgaagtcggcgcggggaggggtagggccaacggcctggacgccccaagggcgggcgcagatcgcggagccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctgggttttgtgtcttcagctgatggatgtgctgactagtgagagtgcttgggccctcccccagcacctagggaaaggcttcccctccccctccggccacaaggtacacaacttttaacttagctcttcccgatgtttgtttgttagtgggaggagtggggagggctggctgtatggcctccagcctacctgttccccctgctcccagggcacatggttgggctgtgtcaacccttagggcctccatggggtcagttgtcccttctcacctcccagctctgtccccatcaggtccctgggtggcacgggaggatggactgacttccaggacctgttgtgtgacaggagctacagcttgggtctccctgcaagaagtctggcacgtctcacctcccccatcccggcccctggtcatctcacagcaaagaagcctcctccctcccgacctgccgccacactggagagggggcacaggggcgggggaggtttcctgttctgtgaaaggccgactccctgactccattcatgcccccccccccagcccctcccttcattcccattccccaacctaaagcctggcccggctcccagctgaatctggtcggaatccacgggctgcagattttccaaaacaatcgttgtatctttattgacttttttttttttttttttctgaatgcaatgactgttttttactcttaaggaaaataaacatcttttagaaacaaaaaaaaaaaa

SEQ ID NO: 21 - DNA, 4922 bp, Homo sapiens
agcagagctgcggccgggggaacccagtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctgggttttgtgtcttcagctgatggatgtgctgactagtgagagtgcttgggccctcccccagcacctagggaaaggcttcccctccccctccggccacaaggtacacaacttttaacttagctcttcccgatgtttgtttgttagtgggaggagtggggagggctggctgtatggcctccagcctacctgttccccctgctcccagggcacatggttgggctgtgtcaacccttagggcctccatggggtcagttgtcccttctcacctcccagctctgtccccatcaggtccctgggtggcacgggaggatggactgacttccaggacctgttgtgtgacaggagctacagcttgggtctccctgcaagaagtctggcacgtctcacctcccccatcccggcccctggtcatctcacagcaaagaagcctcctccctcccgacctgccgccacactggagagggggcacaggggcgggggaggtttcctgttctgtgaaaggccgactccctgactccattcatgcccccccccccagcccctcccttcattcccattccccaacctaaagcctggcccggctcccagctgaatctggtcggaatccacgggctgcagattttccaaaacaatcgttgtatctttattgacttttttttttttttttttctgaatgcaatgactgttttttactcttaaggaaaataaacatcttttagaaacaaaaaaaaaaaa

SEQ ID NO: 22 - DNA, 4056 bp, Homo sapiens
gcggggcgggcgcgccgcagcgctcaacggcttcaaaaatccgccgcgccttgacaggtgaagtcggcgcggggaggggtagggccaacggcctggacgccccaagggcgggcgcagatcgcggagccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctggaaaaaa

(19) United States
(12) Patent Application Publication
(10) Pub. No.: US 2021/0095277 A1
(43) Pub. Date: Apr. 1, 2021

(54) ANTISENSE OLIGONUCLEOTIDES TARGETING SREBP1

(71) Applicant: Roche Innovation Center Copenhagen A/S, Hørsholm (DK)

(72) Inventors: Eva Marie W. LINDHOLM, Malmö (SE); Steffen SCHMIDT, Copenhagen SV (DK)

(73) Assignee: Roche Innovation Center Copenhagen A/S, Hørsholm (DK)

(21) Appl. No.: 15/733,392

(22) PCT Filed: Jan. 16, 2019
(86) PCT No.: PCT/EP2019/051040

§ 371 Date: Jul. 17, 2020

(30) Foreign Application Priority Data
Jan. 18, 2018 (EP) 18152299.6
Jun. 4, 2018 (EP) 18175692.5

(51) Int. Cl. C12N 15/113

(52) U.S. Cl. CPC C12N 15/113; C12N 2310/11; C12N 2310/351; C12N 2310/3231; C12N 2310/315; C12N 2310/341

(57) ABSTRACT

The present invention relates to antisense LNA oligonucleotides (oligomers) complementary to SREBF1 pre-mRNA intron and exon sequences, which are capable of inhibiting the expression of SREBP1 protein. Inhibition of SREBF1 expression is beneficial for a range of medical disorders including cardiovascular disease, type 2 diabetes, fatty liver, metabolic diseases, and cancer.

### FIELD OF INVENTION

The present invention relates to antisense LNA oligonucleotides (oligomers) complementary to SREBF1 pre-mRNA intron and exon sequences, which are capable of inhibiting the expression of SREBP1 protein. Inhibition of SREBF1 expression is beneficial for a range of medical disorders including cardiovascular disease, type 2 diabetes, fatty liver, metabolic diseases, and cancer.

### BACKGROUND

SREBP1, sterol regulatory element binding protein-1 is a protein belonging to the SREBP family of transcription factor. The SREBP family includes three main proteins, SREBP-1a, -1c, and 2, encoded by two genes: SREBF1 and SREBF2. SREBP-1a and -1c (collectively referred to as SREB1 herein) are produced from the same gene through the use of different promoters and alternative splicing.

The SREBP proteins are key regulator of enzymes involved in carbohydrate, triglyceride, fatty acid, and cholesterol metabolism. Overexpression of SREBP is known to be a risk factor for metabolic diseases such as type 2 diabetes, non-alcoholic fatty liver disease, and cardiovascular diseases. Recently SREBP1 over expression has also been implicated in cell growth and so the transcription factor activity has been suggested as causative in cancer (Shao et al., Cell Metab. 2012 Oct. 3; 16(4):414-9), in line with the observation that lipid metabolism is strongly up-regulated in cancer cells.

Small molecule intervention with SREBP signaling has demonstrated an important role for SREBPs in the pathophysiology of the metabolic syndrome (reviewed by Solyal 2015).

Leptin deficient (ob/ob) mice have high hepatic SREBP1c, and SREBP1 knock-out in this mouse strain results in reduced de novo lipogenesis and reduced fatty liver, with no effect on obesity and insulin resistance compared to ob/ob/SREBP1+/+ littermates (Yagahi 2002). Fatostatin, a small molecule inhibitor of SREBP activation, injected daily to ob/ob mice for four weeks, results in reduced liver fat, body weight, and blood glucose compared to controls (Kamisuki 2009), indicating that intervention in adult animals had different effects than knocking out SREBP1 from birth. Note that fatostatin inhibits all SREBP activation and is not specific for SREBP1, increasing the risk for unwanted side effects. Another small molecule blocker of SREBP maturation, betulin, has been reported to improve hyperlipidemia and insulin resistance and reduces atherosclerotic plaques (Tang 2010) in mouse models of disease.

It appears that high SREBP activity is required to maintain a high lipid supply for tumor growth, indicating that normalization of SREBP signaling in tumors is a potential target for anticancer therapy. High SREBP1 signaling independent from systemic regulation, and resulting high de novo lipogenesis, is found in cancers such as prostate cancer, endometrial cancer, and glioblastoma. Fatostatin has been demonstrated to decrease pancreatic cancer cell viability and proliferation (Siqingaowa 2017), and to reduce tumor growth in prostate cancer cell xenografts in mice (Li 2015).

WO2008/011467 refers to putative siRNAs which allegedly mediate RNA interference of SREBP1. US2005/0215504 and US2003/02245151 refer to MOE gapmer antisense compounds and their use to inhibit human or mouse SREBP1 in vitro transfection assays. Compound's which were able to inhibit SREBP1 expression by at least 40% were identified as preferred.

There is a therefore a need for therapeutic agents which can inhibit SREBP1 specifically. We have screened 207 LNA gapmers targeting mouse and human SREBP1 and identified sequences and compounds which are particularly potent and effective to specifically target for SREBP1 antisense in vitro (human and mouse cells, via gymnosis) and in vivo (mouse). The tested compounds were safe and reduced liver, kidney, and adipose SREBP expression. We found that different compounds had different levels of activity in liver, kidney and adipose.

### Objective of the Invention

The inventors have identified particularly effective regions of the SREBP1 transcript (SREBF1) for antisense inhibition in vitro or in vivo, and provides for antisense oligonucleotides, including LNA gapmer oligonucleotides, which target these regions of the SREBF1 premRNA or mature mRNA. The present invention identifies oligonucleotides which inhibit human SREBP1 which are useful in the treatment of a range of medical disorders including cardiovascular disease, type 2 diabetes, fatty liver, metabolic diseases, and cancer.

### STATEMENT OF THE INVENTION

The invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary to SEQ ID NO 14 or SEQ ID NO 15 wherein the antisense oligonucleotide is capable of inhibiting the expression of human SREBP1 in a cell which is expressing human SREBP1. In some embodiments, the antisense oligonucleotide of the invention is capable of inhibiting the expression of SREBP1, such as SREBP1c in a cell, which is expressing said SREBP1.

The invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary to SEQ ID NO 14 or SEQ ID NO 15 wherein the antisense oligonucleotide is capable of inhibiting the expression of human SREBF1 transcript in a cell which is expressing human SREBF1 transcript.

The oligonucleotide of the invention as referred to or claimed herein may be in the form of a pharmaceutically acceptable salt.

The invention provides for a conjugate comprising the oligonucleotide according to the invention, and at least one conjugate moiety covalently attached to said oligonucleotide.

The invention provides for a pharmaceutical composition comprising the oligonucleotide or conjugate of the invention and a pharmaceutically acceptable diluent, solvent, carrier, salt and/or adjuvant.

The invention provides for an in vivo or in vitro method for modulating SREBF1 expression in a target cell which is expressing SREBF1, said method comprising administering an oligonucleotide or conjugate or pharmaceutical composition of the invention in an effective amount to said cell.

The invention provides for a method for treating or preventing a disease comprising administering a therapeutically or prophylactically effective amount of an oligonucleotide, conjugate or the pharmaceutical composition of the invention to a subject suffering from or susceptible to the disease.

In some embodiments, the disease is selected from the group consisting of cardiovascular disease, type 2 diabetes, fatty liver, metabolic diseases, and cancer.

The invention provides for the oligonucleotide, conjugate or the pharmaceutical composition of the invention for use in medicine.

The invention provides for the oligonucleotide, conjugate or the pharmaceutical composition of the invention for use in the treatment or prevention of a disease selected from the group consisting of cardiovascular disease, type 2 diabetes, fatty liver, metabolic diseases, and cancer.

The invention provides for the use of the oligonucleotide, conjugate or the pharmaceutical composition of the invention, for the preparation of a medicament for treatment or prevention of a disease selected from the group consisting of cardiovascular disease, type 2 diabetes, fatty liver, metabolic diseases, and cancer.

### BRIEF DESCRIPTION OF FIGURES

FIG. 1: Testing in vitro efficacy of various antisense oligonucleotides targeting human and mouse SREBF1 mRNA in A549, HeLa and RAW264.7 cell lines at single concentration.

FIG. 2: Comparison of in vitro efficacy for antisense oligonucleotides targeting human SREBF1 mRNA in A549 and HeLa cell lines at single concentration shows good correlation. Two motifs with very efficient targeting are highlighted.

FIG. 3: Testing selected oligonucleotides targeting human (and mouse) SREBF1 mRNA in vitro for concentration dependent potency and efficacy in A549 cell line.

FIG. 4: Testing selected oligonucleotides targeting human (and mouse) SREBF1 mRNA in vitro for concentration dependent potency and efficacy in HeLa cell line.

FIG. 5: Testing selected oligonucleotides targeting (human and) mouse SREBF1 mRNA in vitro for concentration dependent potency and efficacy in RAW264.7 cell line.

FIG. 6: Testing selected oligonucleotides targeting mouse SREBF1 mRNA in vitro for concentration dependent potency and efficacy in RAW264.7 cell line.

FIG. 7: Mouse in vivo efficacy: remaining SREBF1 mRNA transcript in mouse tissues after 16 days of treatment, Intravenous IV (tail vein).

### DEFINITIONS

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl and propyl.

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl, cyclohexyl, cycloheptyl and cyclooctyl, more particularly cyclopropyl and cyclobutyl. A particular example of “cycloalkyl” is cyclopropyl.

The term “alkoxy”, alone or in combination, signifies a group of the formula alkyl-O— in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy and ethoxy. Methoxyethoxy is a particular example of “alkoxyalkoxy”.

The term “oxy”, alone or in combination, signifies the —O— group.

The term “alkenyl”, alone or in combination, signifies a straight-chain or branched hydrocarbon residue comprising an olefinic bond and up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl and isobutenyl.

The term “alkynyl”, alone or in combination, signifies a straight-chain or branched hydrocarbon residue comprising a triple bond and up to 8, preferably up to 6, particularly preferred up to 4 carbon atoms.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl or trifluoromethyl. Fluoromethyl, difluoromethyl and trifluoromethyl are particular “haloalkyl”.

The term “halocycloalkyl”, alone or in combination, denotes a cycloalkyl group as defined above substituted with at least one halogen, particularly substituted with one to five halogens, particularly one to three halogens. Particular example of “halocycloalkyl” are halocyclopropyl, in particular fluorocyclopropyl, difluorocyclopropyl and trifluorocyclopropyl.

The terms “hydroxyl” and “hydroxy”, alone or in combination, signify the —OH group.

The terms “thiohydroxyl” and “thiohydroxy”, alone or in combination, signify the —SH group.

The term “carbonyl”, alone or in combination, signifies the —C(O)— group.

The term “carboxy” or “carboxyl”, alone or in combination, signifies the —COOH group.

The term “amino”, alone or in combination, signifies the primary amino group (—NH₂), the secondary amino group (—NH—), or the tertiary amino group (—N—).

The term “alkylamino”, alone or in combination, signifies an amino group as defined above substituted with one or two alkyl groups as defined above.

The term “sulfonyl”, alone or in combination, means the —SO₂ group.

The term “sulfinyl”, alone or in combination, signifies the —SO— group.

The term “sulfanyl”, alone or in combination, signifies the —S— group.

The term “cyano”, alone or in combination, signifies the —CN group.

The term “azido”, alone or in combination, signifies the —N₃ group.

The term “nitro”, alone or in combination, signifies the NO₂ group.

The term “formyl”, alone or in combination, signifies the —C(O)H group.

The term “carbamoyl”, alone or in combination, signifies the —C(O)NH₂ group.

The term “cabamido”, alone or in combination, signifies the —NH—C(O)—NH₂ group.

The term “aryl”, alone or in combination, denotes a monovalent aromatic carbocyclic mono- or bicyclic ring system comprising 6 to 10 carbon ring atoms, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of aryl include phenyl and naphthyl, in particular phenyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of heteroaryl include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, carbazolyl or acridinyl.

The term “heterocyclyl”, alone or in combination, signifies a monovalent saturated or partly unsaturated mono- or bicyclic ring system of 4 12, in particular 4-9, ring atoms, comprising 1, 2, 3 or 4 ring heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples for monocyclic saturated heterocyclyl are azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl or dihydropyranyl.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

If one of the starting materials or compounds of the invention contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”.

In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkages

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleosides, which according to EP2 742 135 may for example be tolerated in an otherwise DNA phosphorothioate the gap region.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous nucleotide sequence, at a given position of a separate nucleic acid molecule (e.g. the target nucleic acid). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch.

Preferably, insertions and deletions are not allowed in the calculation of % complementarity of a contiguous nucleotide sequence.

The term “fully complementary”, refers to 100% complementarity.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

Target Nucleic Acid

According to the present invention, the target nucleic acid is a nucleic acid which encodes mammalian SREBP1 and may for example be a gene, a SREBF1 RNA, a mRNA, a pre-mRNA, a mature mRNA or a cDNA sequence. The target may therefore be referred to as an SREBP1 target nucleic acid.

Suitably, the target nucleic acid encodes anSREBP1 protein, in particular mammalian SREBP1, such as human SREBP1a or SREBP1c, such as the human SREBP1 encoding pre-mRNA or mRNA sequences provided herein as SEQ ID NO 19, 20, 21 or 22.

In some embodiments, the target nucleic acid is selected from the group consisting of SEQ ID NO: 19 or 20 or naturally occurring variants thereof (e.g. SREBF1 sequences encoding a mammalian SREBP1 protein).

If employing the oligonucleotide of the invention in research or diagnostics the target nucleic acid may be a cDNA or a synthetic nucleic acid derived from DNA or RNA.

For in vivo or in vitro application, the oligonucleotide of the invention is typically capable of inhibiting the expression of the SREBF1 target nucleic acid in a cell which is expressing the SREBF1 target nucleic acid. The contiguous sequence of nucleobases of the oligonucleotide of the invention is typically complementary to the SREBF1 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid is a messenger RNA, such as a mature mRNA or a pre-mRNA which encodes mammalian SREBP1 protein, such as human SREBP1, e.g. the human SREBF1 pre-mRNA sequence, such as that disclosed as SEQ ID NO 19, or SREBF1 mature mRNA, such as that disclosed as SEQ ID NO 20, 21 or 22. SEQ ID NOs 19-22 are DNA sequences—it will be understood that target RNA sequences have uracil (U) bases in place of the thymidine bases (T).

| Target Nucleic Acid | NCBI Sequence | Sequence ID |
|---|---|---|
| SREBF1 Homo sapiens | NG_029029.1 | SEQ ID NO 19 |
| pre- mRNA, | | |
| SREBF1 Homo sapiens | NM_001005291.2 | SEQ ID NO 20 |
| mRNA, transcript variant 1 | | |
| SREBF1 Homo sapiens | NM_004176.4 | SEQ ID NO 21 |
| mRNA, transcript variant 2 | | |
| SREBF1 Homo sapiens | NM_001321096.2 | SEQ ID NO 22 |
| mRNA, transcript variant 3 | | |

In some embodiments, the oligonucleotide of the invention targets SEQ ID NO 19.

In some embodiments, the oligonucleotide of the invention targets SEQ ID NO 20.

In some embodiments, the oligonucleotide of the invention targets SEQ ID NO 21.

In some embodiments, the oligonucleotide of the invention targets SEQ ID NO 22.

In some embodiments, the oligonucleotide of the invention targets SEQ ID NO 19 and at least one of, such as two or three of SEQ ID NO 20, 21 and 22.

In some embodiments, the oligonucleotide of the invention targets SEQ ID NO 19, 20, 21 and 22.

In some embodiments, the oligonucleotide of the invention targets SEQ ID NO 19, 20, and 21.

In some embodiments, the oligonucleotide of the invention targets SEQ ID NO 19, 20, and 22.

Target Sequence

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid which is complementary to the contiguous nucleotide sequence of the oligonucleotide of the invention. In some embodiments the target sequence is longer than the complementary sequence of a single oligonucleotide, and may, for example represent a preferred region of the target nucleic acid which may be targeted by several oligonucleotides of the invention.

The oligonucleotide of the invention comprises a contiguous nucleotide sequence which is complementary to or hybridizes to the target nucleic acid, such as a sub-sequence of the target nucleic acid, such as a target sequence described herein.

The oligonucleotide comprises a contiguous nucleotide sequence which are complementary to a target sequence present in the target nucleic acid molecule. The contiguous nucleotide sequence (and therefore the target sequence) comprises of at least 10 contiguous nucleotides, such as 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

Target Cell

The term a “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell.

In preferred embodiments the target cell expresses SREBF1 mRNA, such as the SREBF1 pre-mRNA, e.g. SEQ ID NO 19, or SREBF1 mature mRNA (e.g. SEQ ID NO 20, 21 or 22).

The poly A tail of SREBF1 mRNA is typically disregarded for antisense oligonucleotide targeting.

Naturally Occurring Variant

The term “naturally occurring variant” refers to variants of SREBF1 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

The Homo sapiens SREBF1 gene is located at chromosome 17, 17811349 . . . 17837017, complement (NC_000017.11, Gene ID 6720).

In some embodiments, the naturally occurring variants have at least 95% such as at least 98% or at least 99% homology to a mammalian SREBF1 target nucleic acid, such as a target nucleic acid selected form the group consisting of SEQ ID NO 19, 20, 21 or 22. In some embodiments the naturally occurring variants have at least 99% homology to the human SREBF1 target nucleic acid of SEQ ID NO: 19.

Modulation of Expression

The term “modulation of expression” as used herein is to be understood as an overall term for an oligonucleotide's ability to alter the amount of SREBP1 protein or SREBF1 mRNA when compared to the amount of SREBP1 or SREBF1 mRNA prior to administration of the oligonucleotide. Alternatively modulation of expression may be determined by reference to a control experiment. It is generally understood that the control is an individual or target cell treated with a saline composition or an individual or target cell treated with a non-targeting oligonucleotide (mock).

One type of modulation is an oligonucleotide's ability to inhibit, down-regulate, reduce, suppress, remove, stop, block, prevent, lessen, lower, avoid or terminate expression of SREBP1, e.g. by degradation of SREBF1 mRNA.

High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

2′ Sugar Modified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

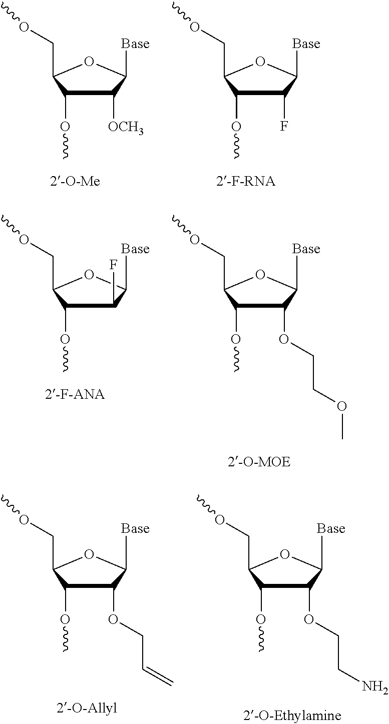

In relation to the present invention 2′ substituted does not include 2′ bridged molecules like LNA.

Locked Nucleic Acids (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

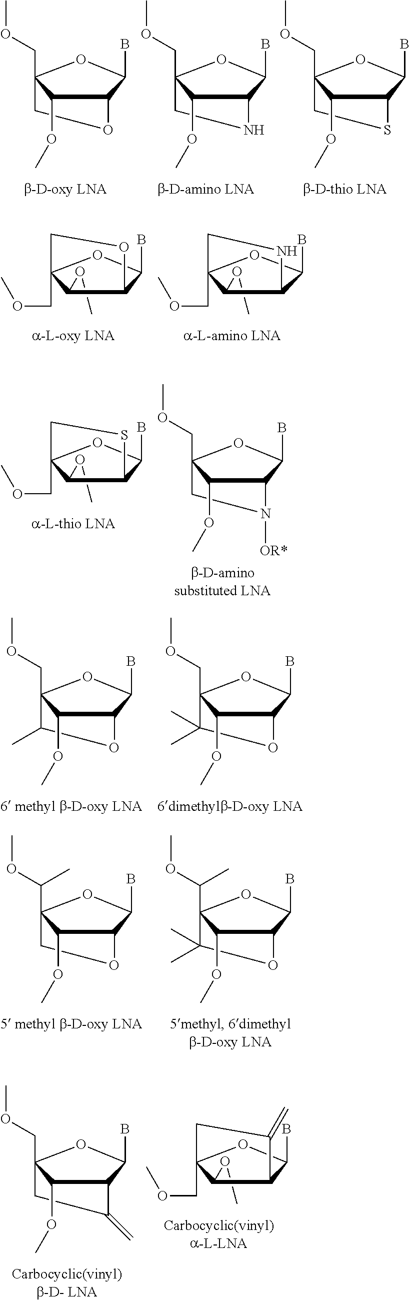

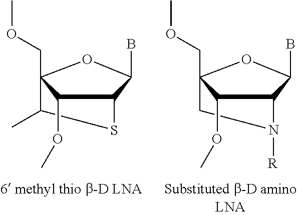

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/1/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

Gapmer

The antisense oligonucleotide of the invention, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ′5->3′ orientation.

The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

Regions F, G and F′ are further defined below and can be incorporated into the F-G-F′ formula.

Gapmer—Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. One or more cytosine (C) DNA in the gap region may in some instances be methylated (e.g. when a DNA c is followed by a DNA g) such residues are either annotated as 5-methyl-cytosine (^{me}C). In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Region G—“Gap-Breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region. Exemplary designs for gap-breaker oligonucleotides include

F₁₋₈-[D₃₋₄-E₁-D₃₋₄]₋F′₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Er Dm], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length. In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer—Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside. It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides. In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]_₅-[region G]-[LNA]₁₋₅, wherein region G is as defined in the Gapmer region G definition.

MOE Gapmers

A MOE gapmers is a gapmer wherein regions F and F′ consist of MOE nucleosides. In some embodiments the MOE gapmer is of design [MOE]_₈-[Region G]-[MOE]₁₋₈, such as [MOE]₂₋₇-[Region G]₅₋₁₆-[MOE]₂₋₇, such as [MOE]₈₋₆-[Region G]-[MOE]₃₆, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

Alternating Flank Gapmers

Oligonucleotides with alternating flanks are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and there is at least one DNA nucleoside positioned between the 5′ and 3′ most LNA nucleosides of region F or F′ (or both region F and F′).

Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein. The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence or gapmer region F-G-F′ (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. DNA phosphodiester containing biocleavable linkers are described in more detail in WO 2014/076195 (hereby incorporated by reference)—see also region D′ or D″ herein.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B—C, A-B—Y—C, A-Y—B—C or A-Y—C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

The invention relates to oligonucleotides, such as antisense oligonucleotides, targeting SREBF1 expression.

The oligonucleotides of the invention targeting SREBF1 are capable of hybridizing to and inhibiting the expression of a SREBF1 target nucleic acid in a cell which is expressing the SREBF1 target nucleic acid.

The SREBF1 target nucleic acid may be a mammalian SREBF1 mRNA or premRNA, such as a human SREBF1 mRNA or premRNA, for example a premRNA or mRNA originating from the Homo sapiens sterol regulatory element binding transcription factor 1 (SREBF1), RefSeqGene on chromosome 17, exemplified by NCBI Reference Sequence: NG_029029.1 (SEQ ID NO 19).

The human SREBF1 pre-mRNA is encoded on Homo sapiens Chromosome 17, NC_000017.11 (17811349 . . . 17837017, complement). GENE ID=6720 (SREBF1).

A mature human mRNA target sequence is illustrated herein by the cDNA sequences SEQ ID NO: 20, 21 or 22.

The oligonucleotides of the invention are capable of inhibiting the expression of SREBF1 target nucleic acid, such as the SREBF1 mRNA, in a cell which is expressing the target nucleic acid, such as the SREBF1 mRNA.

In some embodiments, the oligonucleotides of the invention are capable of inhibiting the expression of SREBF1 target nucleic acid in a cell which is expressing the target nucleic acid, so to reduce the level of SREBF1 target nucleic acid (e.g. the mRNA) by at least 50%, at least 60%, at least 70%, at least 80%, or at least 90% inhibition compared to the expression level of the SREBF1 target nucleic acid (e.g. the mRNA) in the cell. Suitably the cell is selected from the group consisting of A549, HeLa and RAW264.7 cells. Example 1 provides a suitable assay for evaluating the ability of the oligonucleotides of the invention to inhibit the expression of the target nucleic acid. Suitably the evaluation of a compounds ability to inhibit the expression of the target nucleic acid is performed in vitro, such a gymnotic in vitro assay, for example as according to Example 1.

An aspect of the present invention relates to an antisense oligonucleotide, such as an LNA antisense oligonucleotide gapmer which comprises a contiguous nucleotide sequence of 10 to 30 nucleotides in length with at least 90% complementarity, such as is fully complementary to SEQ ID NO 19 or SEQ ID NO 20.

An aspect of the present invention relates to an antisense oligonucleotide, such as an LNA antisense oligonucleotide gapmer which comprises a contiguous nucleotide sequence of 10 to 30 nucleotides in length with at least 90% complementarity, such as is fully complementary to SEQ ID NO 21 or SEQ ID NO 22.

In some embodiments, the oligonucleotide comprises a contiguous sequence of 10-30 nucleotides, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

The inventors have identified particularly effective sequences of the SREBF1 target nucleic acid which may be targeted by the oligonucleotide of the invention.

In some embodiments the target sequence is SEQ ID NO 14.

In some embodiments the target sequence is SEQ ID NO 15.

In some embodiments the target sequence is SEQ ID NO 16.

In some embodiments the target sequence is SEQ ID NO 17.

In some embodiments the target sequence is SEQ ID NO 18.

SEQ ID NO 14:
CTCCATTGAAGATGTACCCGTCCATGCCCG
(19, 20, 21, 22
SEQ ID NO 15:
CTGAATGCAATGACTGTTTTTTACTCTTAAGGAAAATAAACATCT
(19, 20, 21)
SEQ ID NO 16:
AAGATGTACCCGTCC
(19, 20, 22)
SEQ ID NO 17:
CTGAATGCAATGACTGTT
(19, 20, 21,
SEQ ID NO 18:
CTTAAGGAAAATAAACATCT
(19, 20, 21)

(numbers in brackets refer to the SEQ ID of SREBF1 premRNA or mRNA transcripts in which the target sequence is found).

In some embodiments, the oligonucleotide of the invention comprises a contiguous nucleotides sequence of 12-24, such as 13, 14, 15, 16, 17, 18, 19, 20, 21, 22, or 23, contiguous nucleotides in length, wherein the contiguous nucleotide sequence is fully complementary to SEQ ID NO 14.

In some embodiments, the oligonucleotide of the invention comprises a contiguous nucleotides sequence of 12-24, such as 13, 14, 15, 16, 17, 18, 19, 20, 21, 22, or 23, contiguous nucleotides in length, wherein the contiguous nucleotide sequence is fully complementary to SEQ ID NO 15.

In some embodiments, the antisense oligonucleotide of the invention comprises a contiguous nucleotides sequence of 12-15, such as 13, or 14, 15 contiguous nucleotides in length, wherein the contiguous nucleotide sequence is fully complementary to SEQ ID NO 16.

In some embodiments, the antisense oligonucleotide of the invention comprises a contiguous nucleotides sequence of 12-18, such as 13, 14, 15, 16, or 17, contiguous nucleotides in length, wherein the contiguous nucleotide sequence is fully complementary to SEQ ID NO 17.

In some embodiments, the antisense oligonucleotide of the invention comprises a contiguous nucleotides sequence of 12-20, such as 13, 14, 15, 16, 17, 18 or 19 contiguous nucleotides in length, wherein the contiguous nucleotide sequence is fully complementary to SEQ ID NO 18.

In some embodiments, the antisense oligonucleotide of the invention or the contiguous nucleotide sequence thereof is a gapmer, such as an LNA gapmer, a mixed wing gapmer, or an alternating flank gapmer.

In some embodiments, the antisense oligonucleotide according to the invention, comprises a contiguous nucleotide sequence of at least 10 contiguous nucleotides, such as at least 12 contiguous nucleotides, such as at least 13 contiguous nucleotides, such as at least 14 contiguous nucleotides, such as at least 15 contiguous nucleotides, which is fully complementary to SEQ ID NO 14.

In some embodiments, the antisense oligonucleotide according to the invention, comprises a contiguous nucleotide sequence of at least 10 contiguous nucleotides, such as at least 12 contiguous nucleotides, such as at least 13 contiguous nucleotides, such as at least 14 contiguous nucleotides, such as at least 15 contiguous nucleotides, which is fully complementary to SEQ ID NO 15.

In some embodiments, the antisense oligonucleotide according to the invention, comprises a contiguous nucleotide sequence of at least 10 contiguous nucleotides, such as at least 12 contiguous nucleotides, such as at least 13 contiguous nucleotides, such as at least 14 contiguous nucleotides, such as at least 15 contiguous nucleotides, which is fully complementary to SEQ ID NO 16.

In some embodiments, the antisense oligonucleotide according to the invention, comprises a contiguous nucleotide sequence of at least 10 contiguous nucleotides, such as at least 12 contiguous nucleotides, such as at least 13 contiguous nucleotides, such as at least 14 contiguous nucleotides, such as at least 15 contiguous nucleotides, which is fully complementary to SEQ ID NO 17.

In some embodiments, the antisense oligonucleotide according to the invention, comprises a contiguous nucleotide sequence of at least 10 contiguous nucleotides, such as at least 12 contiguous nucleotides, such as at least 13 contiguous nucleotides, such as at least 14 contiguous nucleotides, such as at least 15 contiguous nucleotides, which is fully complementary to SEQ ID NO 18.

In some embodiments the contiguous nucleotide sequence of the antisense oligonucleotide according to the invention is less than 20 nucleotides in length. In some embodiments the contiguous nucleotide sequence of the antisense oligonucleotide according to the invention is 12-24 nucleotides in length. In some embodiments the contiguous nucleotide sequence of the antisense oligonucleotide according to the invention is 12-22 nucleotides in length.

In some embodiments the contiguous nucleotide sequence of the antisense oligonucleotide according to the invention is 12-20 nucleotides in length. In some embodiments the contiguous nucleotide sequence of the antisense oligonucleotide according to the invention is 12-18 nucleotides in length. In some embodiments the contiguous nucleotide sequence of the antisense oligonucleotide according to the invention is 12-16 nucleotides in length.

Advantageously, in some embodiments all of the internucleoside linkages between the nucleosides of the contiguous nucleotide sequence are phosphorothioate internucleoside linkages.

In some embodiments, the contiguous nucleotide sequence is fully complementary to SEQ ID NO 14.

In some embodiments, the contiguous nucleotide sequence is fully complementary to SEQ ID NO 15.

In some embodiments, the contiguous nucleotide sequence is fully complementary to SEQ ID NO 16.

In some embodiments, the contiguous nucleotide sequence is fully complementary to SEQ ID NO 17 or SEQ ID NO 18.

In some embodiments, the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region between 5 and 16 nucleosides which are capable of recruiting RNaseH.

In some embodiments, the sugar modified nucleosides of region F and F′ are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

In some embodiments, region G comprises 5-16 contiguous DNA nucleosides.

In some embodiments, wherein the antisense oligonucleotide is a gapmer oligonucleotide, such as an LNA gapmer oligonucleotide.

In some embodiments, the LNA nucleosides are beta-D-oxy LNA nucleosides.

In some embodiments, the internucleoside linkages between the contiguous nucleotide sequence are phosphorothioate internucleoside linkages.

Sequence Motifs and Compounds of the Invention

| | | Sequence | | | Compound |
|---|---|---|---|---|---|
| Target | | motif | Compound | | LNA |
| region | Sequence Motif | SEQ ID | ID | Compound | pattern |
| SEQ ID | GACGGGTACATCTT | 1 | CMP 1,1 | GACgggtacatCTT | 3-8-3 |
| NO 14 | | | | | |
| SEQ ID | GGACGGGTACATCTT | 2 | CMP 2,1 | GGA^{m}cgggtacatcTT | 3-10-2 |
| NO 14 | | | | | |
| SEQ ID | CAGTCATTGCATTCAG | 3 | CMP 3,1 | CAgtcattgcattCAG | 2-10-3 |
| NO 15 | | | | | |
| SEQ ID | ACAGTCATTGCATTCAG | 4 | CMP 4,1 | ACagtcattgcattCAG | 2-12-3 |
| NO 15 | CACTGTCTTGGTTGTTGAT | 5 | CMP 5,1 | CActgtcttggttgttgAT | |
| | CTGTCTTGGTTGTTGAT | 6 | CMP 6,1 | CTgtcttggttgttgAT | 2-15-2 |
| SEQ ID | AACAGTCATTGCATTCA | 7 | CMP 7,1 | AACagtcattgcattCA | 3-12-2 |
| NO 15 | AGATGTTTATTTTCCTTAAG | 8 | CMP 8,1 | AGATgtttattttccttaAG | |
| SEQ ID | AAGACAGCAGATTTATTC | 9 | CMP 9,1 | AAGAcagcagatttatTC | 4-14-2 |
| NO 15 | CAGCAGATTTATTCAGC | 10 | CMP 10,1 | CAgcagatttattcAGC | |

In the compound column, capital letters are beta-D-oxy LNA nucleosides, and LNA C are all 5-methyl C, lower case letters are DNA nucleosides, and a superscript m before a lower case c represent a 5-methyl cytosine DNA nucleoside, and all internucleoside linkages are phosphorothioate internucleoside linkages.

The invention provides antisense oligonucleotides according to the invention, such as antisense oligonucleotides 12-24, such as 12-18 in length, nucleosides in length wherein the antisense oligonucleotide comprises a contiguous nucleotide sequence comprising at least 12, such as at least 14, such as at least 15 contiguous nucleotides present in SEQ ID NO 1 or 2.

The invention provides antisense oligonucleotides according to the invention, such as antisense oligonucleotides 12-24 nucleosides in length, such as 12-18 in length, wherein the antisense oligonucleotide comprises a contiguous nucleotide sequence comprising at least 12, such as at least 13, such as at least 14, such as at least 15 contiguous nucleotides present in SEQ ID NO 3 or 4 or 7.

The invention provides antisense oligonucleotides according to the invention, such as antisense oligonucleotides 12-24 nucleosides in length, such as 12-18 in length, wherein the antisense oligonucleotide comprises a contiguous nucleotide sequence comprising at least 12, such as at least 13, such as at least 14, such as at least 15 contiguous nucleotides present in SEQ ID NO 8.

The invention provides LNA gapmers according to the invention comprising or consisting of a contiguous nucleotide sequence selected from SEQ ID NO 1-10.

The invention provides antisense oligonucleotides selected from the group consisting of: GACgggtacatCTT, GGAcgggtacatcTT, CAgtcattgcattCAG, ACagtcattgcattCAG, CActgtcttggttgttgAT, CTgtcttggttgttgAT, AACagtcattgcattCA, ΔGATgtttattttccttaAG, AAGAcagcagatttatTC, CAgcagatttattcAGC; wherein a capital letter is a LNA nucleoside, and a lower case letter is a DNA nucleoside. In some embodiments all internucleoside linkages in contiguous nucleoside sequence are phosphorothioate internucleoside linkages. Optionally LNA cytosine may be 5-methyl cytosine. Optionally DNA cytosine may be 5-methyl cytosine.

The invention provides antisense oligonucleotides selected from the group consisting of: GACgggtacatCTT, GGAcgggtacatcTT, CAgtcattgcattCAG, ACagtcattgcattCAG, CActgtcttggttgttgAT, CTgtcttggttgttgAT, AACagtcattgcattCA, ΔGATgtttattttccttaAG, AAGAcagcagatttatTC, CAgcagatttattcAGC; wherein a capital letter is a beta-D-oxy-LNA nucleoside, and a lower case letter is a DNA nucleoside. In some embodiments all internucleoside linkages in contiguous nucleoside sequence are phosphorothioate internucleoside linkages. Optionally LNA cytosine may be 5-methyl cytosine. Optionally DNA cytosine may be 5-methyl cytosine.

The invention provides antisense oligonucleotides selected from the group consisting of: GACgggtacatCTT, GGAcgggtacatcTT, CAgtcattgcattCAG, ACagtcattgcattCAG, CActgtcttggttgttgAT, CTgtcttggttgttgAT, AACagtcattgcattCA, ΔGATgtttattttccttaAG, AAGAcagcagatttatTC, CAgcagatttattcAGC; wherein a capital letter is a beta-D-oxy-LNA nucleoside, wherein all LNA cytosinese are 5-methyl cytosine, and a lower case letter is a DNA nucleoside, wherein all internucleoside linkages in contiguous nucleoside sequence are phosphorothioate internucleoside linkages, and optionally DNA cytosine may be 5-methyl cytosine.

Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

GalNAc Conjugates

In some embodiments, the conjugate moiety comprises or is an asialoglycoprotein receptor targeting moiety, which may include, for example galactose, galactosamine, N-formyl-galactosamine, Nacetylgalactosamine, N-propionyl-galactosamine, N-n-butanoyl-galactosamine, and N-isobutanoylgalactos-amine. In some embodiments the conjugate moiety comprises a galactose cluster, such as N-acetylgalactosamine trimer. In some embodiments, the conjugate moiety comprises a GalNAc (N-acetylgalactosamine), such as a mono-valent, di-valent, tri-valent of tetra-valent GalNAc. Trivalent GalNAc conjugates may be used to target the compound to the liver (see e.g. U.S. Pat. No. 5,994,517 and Hangeland et al., Bioconjug Chem. 1995 November-December; 6(6):695-701, WO2009/126933, WO2012/089352, WO2012/083046, WO2014/118267, WO2014/179620, & WO2014/179445), see also the exemplified example in FIG. 8. These GalNAc references and the specific conjugates used therein are hereby incorporated by reference.

In some embodiments the conjugate of the invention comprises the trilavent GalNAc conjugate disclosed in FIG. 8.

Exemplary conjugates of the invention include:

5′-GN2-C6ₒcₒaₒGₛAₛCₛgₛgₛgₛtₛaₛcₛaₛtₛCₛTₛT;
5′-GN2-C6ₒcₒaₒGₛGₛAₛcₛgₛgₛgₛtₛaₛcₛaₛtₛcₛTₛT;
5′-GN2-C6ₒcₒaₒCₛAₛgₛtₛcₛaₛtₛtₛgₛcₛaₛtₛtₛCₛAₛG;
and
5′-GN2-C6ₒcₒaₒCₛCₛtₛaₛgₛtₛaₛaₛgₛcₛCₛAₛCₛG;

wherein a capital letter represents a beta-D-oxy LNA nucleoside, a lower case letter represents a DNA nucleoside, wherein each LNA cytosine is 5-methyl cytosine, and mc is 5-methyl cytosine DNA, and wherein subscript s represents a phosphorothioate internucleoside linkage, and a subscript o represents a phosphodiester internucleoside linkage, and GN2-C6 is a 5′ conjugate of formula:

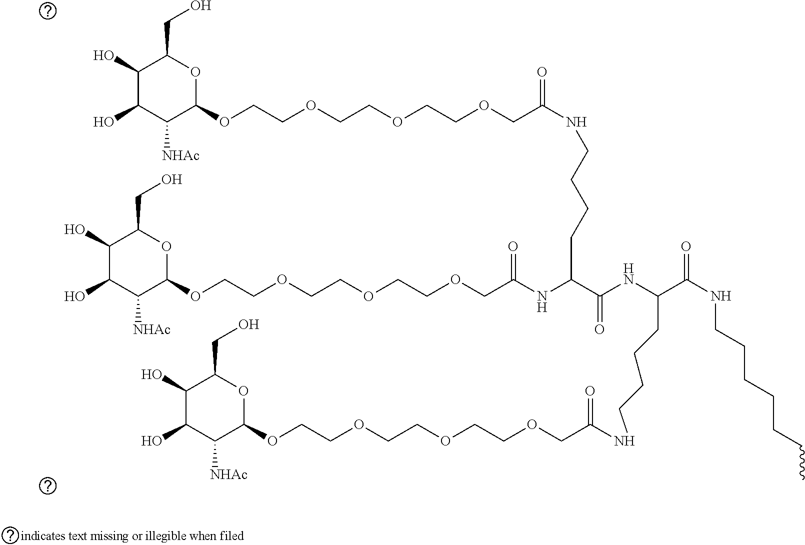

wherein the wavy line represents the covalent bond to the phosphodiester linkage at the 5′ end of the oligonucleotide.

Conjugate Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety to an oligonucleotide (e.g. the termini of region A or C). In some embodiments of the invention the conjugate or oligonucleotide conjugate of the invention may optionally, comprise a linker region which is positioned between the oligonucleotide and the conjugate moiety. In some embodiments, the linker between the conjugate and oligonucleotide is biocleavable.

Biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Conjugates may also be linked to the oligonucleotide via non biocleavable linkers, or in some embodiments the conjugate may comprise a non-cleavable linker which is covalently attached to the biocleavable linker. Linkers that are not necessarily biocleavable but primarily serve to covalently connect a conjugate moiety to an oligonucleotide or biocleavable linker. Such linkers may comprise a chain structure or an oligomer of repeating units such as ethylene glycol, amino acid units or amino alkyl groups. In some embodiments the linker (region Y) is an amino alkyl, such as a C2-C36 amino alkyl group, including, for example C6 to C12 amino alkyl groups. In some embodiments the linker (region Y) is a C6 amino alkyl group. Conjugate linker groups may be routinely attached to an oligonucleotide via use of an amino modified oligonucleotide, and an activated ester group on the conjugate group.

Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

Applications

The oligonucleotides of the invention may be utilized as research reagents for, for example, diagnostics, therapeutics and prophylaxis.

In research, such oligonucleotides may be used to specifically modulate the synthesis of SREBP1 protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

The present invention provides an in vivo or in vitro method for modulating SREBF1 expression in a target cell which is expressing SREBF1, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal.

In diagnostics the oligonucleotides may be used to detect and quantitate SREBF1 expression in cell and tissues by northern blotting, in-situ hybridisation or similar techniques.

For therapeutics, an animal or a human, suspected of having a disease or disorder, which can be treated by modulating the expression of SREBF1 The invention provides methods for treating or preventing a disease, comprising administering a therapeutically or prophylactically effective amount of an oligonucleotide, an oligonucleotide conjugate or a pharmaceutical composition of the invention to a subject suffering from or susceptible to the disease.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment of a disorder as referred to herein, or for a method of the treatment of as a disorder as referred to herein.

The disease or disorder, as referred to herein, is associated with expression of SREBF1. In some embodiments disease or disorder may be associated with a mutation in the SREBF1 gene. Therefore, in some embodiments, the target nucleic acid is a mutated form of the SREBF1 sequence.

The methods of the invention are preferably employed for treatment or prophylaxis against diseases caused by abnormal levels and/or activity of SREBF1.

The invention further relates to use of an oligonucleotide, oligonucleotide conjugate or a pharmaceutical composition as defined herein for the manufacture of a medicament for the treatment of abnormal levels and/or activity of SREBF1.

In one embodiment, the invention relates to oligonucleotides, oligonucleotide conjugates or pharmaceutical compositions for use in the treatment of diseases or disorders selected from cardiovascular disease, type 2 diabetes, fatty liver, metabolic diseases, and cancer.

Administration

The oligonucleotides or pharmaceutical compositions of the present invention may be administered topical or enteral or parenteral (such as, intravenous, subcutaneous, intra-muscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

The work leading to this invention has received funding from the European Union Seventh Framework Programme [FP7-2007-2013] under grant agreement “HEALTH-F2-2013-602222” (Athero-Flux).

### EXAMPLES

### Example 1: Testing In Vitro Efficacy of Antisense Oligonucleotides Targeting Human (and Mouse) SREBF1 mRNA in A549, HeLa (and RAW264.7) Cell Lines at Single Concentration

A549, HeLa and RAW264.7 cell lines were purchased from ATCC and maintained as recommended by the supplier in a humidified incubator at 37° C. with 5% CO₂. For assays, 3000 cells/well (A549; HeLa) or 2500 cells/well (RAW264.7) were seeded in a 96 multi well plate in culture media. Cells were incubated for 24 hours before addition of oligonucleotides dissolved in PBS. Final concentration of oligonucleotides: 25 μM. 3 days after addition of oligonucleotides, the cells were harvested. RNA was extracted using the PureLink Pro 96 RNA Purification kit (Thermo Fisher Scientific) according to the manufacturer's instructions and eluated in 50 μl water. The RNA was subsequently diluted 10 times with DNase/RNase free Water (Gibco) and heated to 90° C. for one minute.

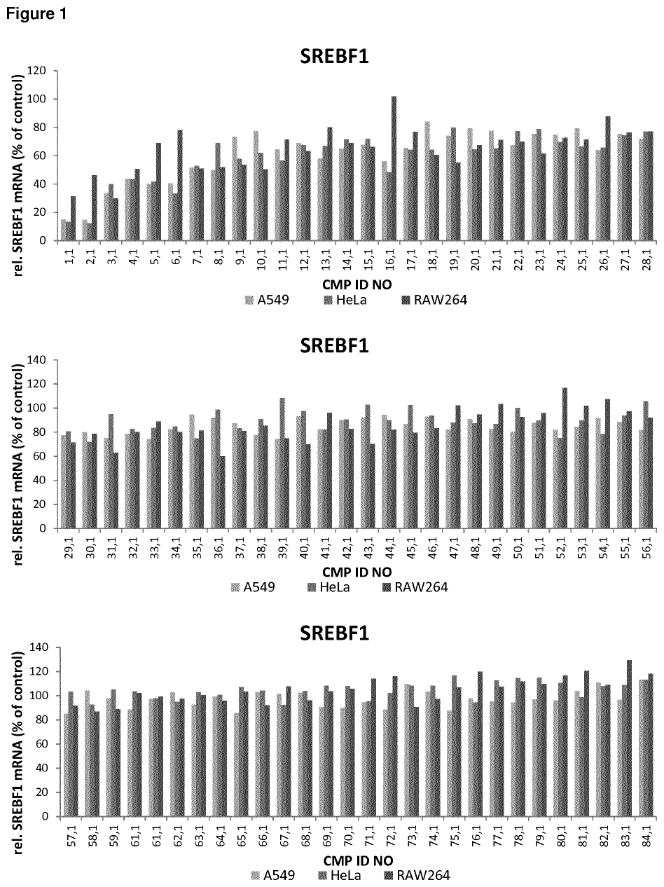

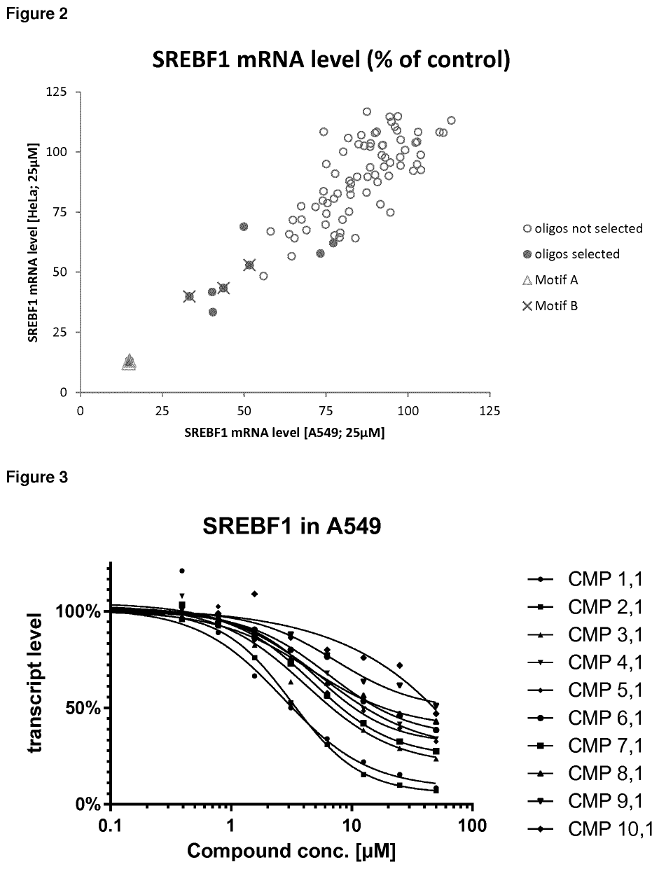

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: SREBF1, Hs01088679_g1 (Mm00550338_m1) [FAM-MGB] and endogenous control GAPDH, Hs99999905_m1 (Mm99999915_g1) [VIC-MGB]. All primer sets were purchased from Thermo Fisher Scientific. The relative NFKB1 mRNA expression level in the table is shown as percent of control (PBS-treated cells). 84 LNA gapmer antisense oligonucleotides targeting the human SREBP1 transcript (premRNA or mRNA) we designed, and were assayed in the above assay—the SREBP1 mRNA levels from cells treated with a selection of the compounds are shown in FIG. 1 and FIG. 2, evaluated in human HeLa and A549 cell lines. From the initial library screen we identified 2 motifs on the SREBP1 human transcript which provided surprisingly effective and potent compounds in the cell lines tested: Motif A (SEQ ID NO 13), and Motif B (SEQ ID NO 14).

Selected Oligonucleotides Used:

| | | | | rel. mRNA | rel. mRNA | rel. mRNA |
|---|---|---|---|---|---|---|
| SEQ | | CMP | | level in | level in | level in |
| ID | | ID | | A549 at | HeLa at | RAW264 at |
| NO | Motif | NO | Compound | 25 μM | 25 μM | 25 μM |
| 1 | gacgggtacatctt | 1,1 | GACgggtacatCTT | 15 | 13 | 31 |
| 2 | ggacgggtacatctt | 2,1 | GGA^{m}cgggtacatcTT | 15 | 12 | 46 |
| 3 | cagtcattgcattcag | 3,1 | CAgtcattgcattCAG | 33 | 40 | 30 |
| 4 | acagtcattgcattcag | 4,1 | ACagtcattgcattCAG | 44 | 43 | 50 |
| 5 | cactgtcttggttgttgat | 5,1 | CActgtcttggttgttgAT | 40 | 42 | 69 |
| 6 | ctgtcttggttgttgat | 6,1 | CTgtcttggttgttgAT | 41 | 33 | 78 |
| 7 | aacagtcattgcattca | 7,1 | AACagtcattgcattCA | 52 | 53 | 51 |
| 8 | agatgtttattttccttaag | 8,1 | AGATgtttattttccttaAG | 50 | 69 | 52 |
| 9 | aagacagcagatttattc | 9,1 | AAGAcagcagatttatTC | 73 | 58 | 54 |
| 10 | cagcagatttattcagc | 10,1 | CAgcagatttattcAGC | 77 | 62 | 50 |
| 11 | tatatagtcagtcacg | M1,1 | TAtatagtcagtCACG | — | — | 15 |
| 12 | cctagtaagccacg | M2,1 | CCtagtaagcCACG | — | — | 16 |
| 13 | tcctagtaagccacg | M3,1 | TCctagtaagcCACG | — | — | 16 |

For Compounds: Capital letters represent LNA nucleosides (beta-D-oxy LNA nucleosides were used), all LNA cytosines are 5-methyl cytosine, lower case letters represent DNA nucleosides, DNA cytosines preceded with a superscript ^{m} represents a 5-methyl C-DNA nucleoside. All internucleoside linkages are phosphorothioate internucleoside linkages. Compounds M1,1; M2,1 and M3,1 target the mouse SREBP1 transcript only.

### Example 2: Testing In Vitro Potency and Efficacy of Selected Oligonucleotides Targeting Human SREBF1 mRNA in A549 and HeLa Cell Lines Dependend on Concentration

A549 cell line and HeLa cell line was described in Example 1. The assay was performed as described in Example 1. Concentration of oligonucleotides: from 50 μM, half-log dilution, 8 points. 3 days after addition of oligonucleotides, the cells were harvested. RNA extraction and duplex One Step RT-qPCR were performed as described in Example 1. Determination of IC50 values was performed in GraphPad Prism6. The relative SREBFE1 mRNA level at treatment with 50 μM oligonucleotide is shown in the table as percent of control (PBS).

| | | IC50 A549 | mRNA level at Max | IC50 HeLa | mRNA level at Max | IC50 RAW264.7 | mRNA level at Max |
|---|---|---|---|---|---|---|---|
| SEQ ID NO | CMP ID NO | [μM] | KD in A549 | [μM] | KD in HeLa | [μM] | KD in RAW264.7 |
| 1 | 1.1 | 2 | 9 | 2 | 21 | 2.5 | 23 |
| 2 | 2.1 | 3 | 7 | 1.9 | 17 | 1.7 | 64 |
| 3 | 3.1 | 4.1 | 23 | 2.7 | 36 | 2.2 | 35 |
| 4 | 4.1 | 5.3 | 34 | 2.7 | 45 | 1.6 | 44 |
| 5 | 5.1 | 5.1 | 32 | 2.3 | 40 | 0.7 | 91 |
| 6 | 6.1 | 6.4 | 38 | 3.7 | 47 | 1.9 | 53 |
| 7 | 7.1 | 4.7 | 27 | 2.3 | 30 | 1.2 | 71 |
| 8 | 8.1 | 4.5 | 43 | 3.1 | 61 | 0.4 | 70 |
| 9 | 9.1 | 7.3 | 51 | 2.4 | 70 | 2.5 | 64 |
| 10 | 10.1 | n.d. | 47 | 7.4 | 59 | 2.1 | 55 |
| 21 | M1.1 | — | — | — | — | 2.3 | 13 |
| 22 | M2.1 | — | — | — | — | 6.8 | 4 |
| 23 | M3.1 | — | — | — | — | 6.7 | 3 |

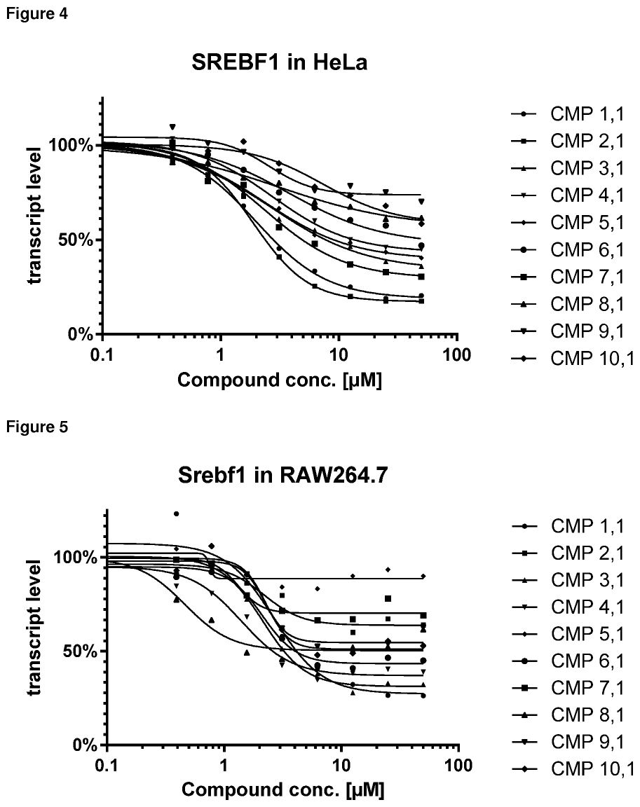

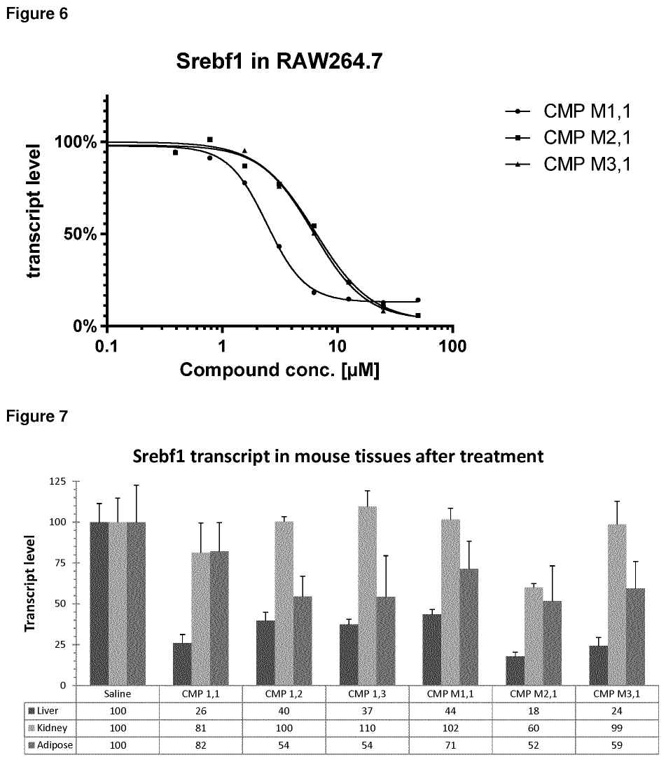

The concentration response curves in A549, HeLa, RAW264.7, are provided as FIGS. 3, 4, and 5 respectively. FIG. 6 provides the concentration response curves from RAW264.7 cells for the three mouse specific Srebf1 targeting compounds.

### Example 3: Mouse In Vivo Efficacy and Tolerance Study, 16 Days of Treatment, Intravenous IV (Tail Vein)

Animals

Experiment was performed on female C57BL/6JBom mice. Five animals were included in each group of the study, including a saline control group.

Compounds and Dosing Procedures

Animals were dosed intravenous (tail vein) with 15 mg/kg compound at day 0, 3, 7, 10, 14 until the study was terminated at day 16.

Euthanasia

At the end of the study (day 16) all mice were euthanized with C02 before tissue samples of liver, kidney and adipose tissue were dissected and snap frozen.

Quantification of Srebf1 RNA Expression

Tissue samples were kept frozen until lysed in MagNA Pure LC RNA Isolation Tissue Lysis Buffer (Product No. 03604721001, Roche) and RNA extraction continued using the MagNA Pure 96 Cellular RNA Large Volume Kit (Product No. 05467535001, Roche) on a MagNA Pure 96 Instrument (Roche) according to the user's manual and RNA diluted to 5 ng/μl in water.

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: Srebf1, Mm00550338_m1 (FAM-MGB) and endogenous control Gapdh, Mm99999915_g1 (VIC-MGB). All primer sets were purchased from Thermo Fisher Scientific. The relative mRNA expression levels are shown as % of saline treated control group (FIG. 7).

1. An antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary to SEQ ID NO 14 or SEQ ID NO 15, wherein the antisense oligonucleotide is capable of inhibiting the expression of human SREBF1 in a cell which is expressing human SREBF1; or a pharmaceutically acceptable salt thereof.

2. The antisense oligonucleotide according to claim 1, wherein the contiguous nucleotide sequence is fully complementary to SEQ ID NO 14 or SEQ ID NO 15.

3. The antisense oligonucleotide according to claim 1, wherein the contiguous nucleotide sequence is fully complementary to SEQ ID NO 16.

4. The antisense oligonucleotide according to claim 1, wherein the contiguous nucleotide sequence is fully complementary to SEQ ID NO 17 or SEQ ID NO 18.

5. The antisense oligonucleotide according to claim 1, wherein the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region of between 5 and 16 nucleosides which are capable of recruiting RNaseH.

6. The antisense oligonucleotide according to claim 5, wherein the sugar modified nucleosides of region F and region F′ are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

7. The antisense oligonucleotide according to claim 5, wherein region G comprises 5-16 contiguous DNA nucleosides.

8. The antisense oligonucleotide according to claim 1, wherein the antisense oligonucleotide is a LNA gapmer oligonucleotide.

9. The antisense oligonucleotide according to claim 6, wherein the LNA nucleosides are beta-D-oxy LNA nucleosides.

10. The antisense oligonucleotide according to claim 1, wherein the internucleoside linkages between the contiguous nucleotide sequence are phosphorothioate internucleoside linkages.

11. The antisense oligonucleotide according to claim 1, wherein the oligonucleotide comprises a contiguous nucleotide sequence selected from the group consisting of: SEQ ID NO 1, SEQ ID NO 2, SEQ ID NO 3, SEQ ID NO 4, SEQ ID NO 7 and SEQ ID NO 8.

12. The antisense oligonucleotide according to claim 1, wherein the oligonucleotide comprises or consists of a contiguous nucleotide sequence selected from:
(SEQ ID NO 1)
GACgggtacatCTT
(SEQ ID NO 2)
GGAcgggtacatcTT
(SEQ ID NO 3)
CAgtcattgcattCAG
(SEQ ID NO 4)
ACagtcattgcattCAG
(SEQ ID NO 7)
AACagtcattgcattCA
(SEQ ID NO 8)
AGATgtttattttccttaAG
wherein a capital letter represents a LNA nucleoside, and a lower case letter represents a DNA nucleoside.

13. The antisense oligonucleotide according to claim 1, wherein the oligonucleotide comprises or consists of a contiguous nucleotide sequence selected from:
(SEQ ID NO 1)
GACgggtacatCTT
(SEQ ID NO 2)
GGA^{m}cgggtacatcTT
(SEQ ID NO 3)
CAgtcattgcattCAG
(SEQ ID NO 4)
ACagtcattgcattCAG
(SEQ ID NO 7)
AACagtcattgcattCA
(SEQ ID NO 8)
AGATgtttattttccttaAG
wherein a capital letter represents a beta-D-oxy LNA nucleoside, a lower case letter represents a DNA nucleoside, wherein each LNA cytosine is 5-methyl cytosine, and ^{m}c is 5-methyl cytosine DNA, and wherein the internucleoside linkages between the nucleosides are phosphorothioate internucleoside linkages.

14. A conjugate comprising the oligonucleotide according to claim 1, and at least one conjugate moiety covalently attached to said oligonucleotide.

15. The conjugate according to claim 14, wherein the conjugate moiety is a trilavent GalNAc conjugate moiety, such as the conjugate moiety of formula 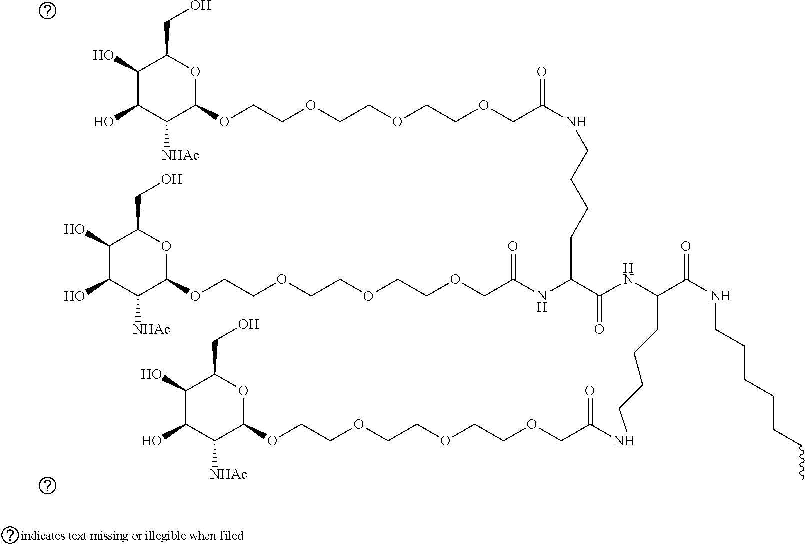
wherein the wavy line represents the covalent bond to the 5′ end of the oligonucleotide.

16. The conjugate according to claim 14, wherein the compound is selected from the group consisting of:
5′-GN2-C6ₒcₒaₒGₛAₛCₛgₛgₛgₛtₛaₛcₛaₛtₛCₛTₛT;
5′-GN2-C6ₒcₒaₒGₛGₛAₛcₛgₛgₛgₛtₛaₛcₛaₛtₛcₛTₛT;
5′-GN2-C6ₒcₒaₒCₛAₛgₛtₛcₛaₛtₛtₛgₛcₛaₛtₛtₛCₛAₛG;
and
5′-GN2-Cₒ6ₒcₒaCₛCₛtₛaₛgₛtₛaₛaₛgₛcₛCₛAₛCₛG;
wherein a capital letter represents a beta-D-oxy LNA nucleoside, a lower case letter represents a DNA nucleoside, wherein each LNA cytosine is 5-methyl cytosine, and ^{m}c is 5-methyl cytosine DNA, and wherein subscript s represents a phosphorothioate internucleoside linkage, and a subscript o represents a phosphodiester internucleoside linkage, and GN2-C6 is a 5′ conjugate of formula: 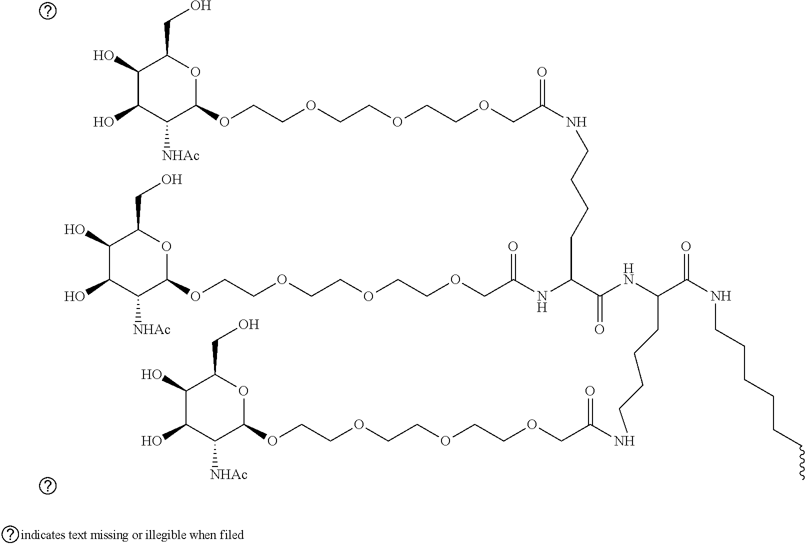
wherein the wavy line represents the covalent bond to the phosphodiester linkage at the 5′ end of the oligonucleotide.

17. A pharmaceutical composition comprising the oligonucleotide of claim 1 and a pharmaceutically acceptable diluent, solvent, carrier, salt and/or adjuvant.

18. An in vivo or in vitro method for modulating SREBF1 expression in a target cell which is expressing SREBF1, said method comprising administering an oligonucleotide of claim 1 in an effective amount to said cell.

19. A method for treating or preventing a disease comprising administering a therapeutically or prophylactically effective amount of an oligonucleotide of claim 1 to a subject suffering from or susceptible to the disease.

20. The method of claim 19, wherein the disease is selected from the group consisting of cardiovascular disease, type 2 diabetes, fatty liver, metabolic diseases, and cancer.

21. The oligonucleotide of claim 1 for use in medicine.

22. The oligonucleotide of claim 1 for use in the treatment or prevention of a disease selected from the group consisting of cardiovascular disease, type 2 diabetes, fatty liver, metabolic diseases, and cancer.

23. Use of the oligonucleotide of claim 1, for the preparation of a medicament for treatment or prevention of a disease selected from the group consisting of cardiovascular disease, type 2 diabetes, fatty liver, metabolic diseases, and cancer.
